(19) United States
(12) Patent Application Publication
(10) Pub. No.: US 2022/0411799 A1
(43) Pub. Date: Dec. 29, 2022

(54) DNMT1-Specific Aptamers and Production and Uses Thereof

(71) Applicants: National University of Singapore, Singapore (SG); Beth Israel Deaconess Medical Center, Inc., Boston, MA (US); Consiglio Nazionale Delle Ricerche, Roma (IT)

(72) Inventors: Daniel Geoffrey Tenen, Singapore (SG); Carla Lucia Esposito, Naples (IT); Annalisa Di Ruscio, Boston, MA (US); Vittorio De Franciscis, Naples (IT); Alexander K. Ebralidze, Boston, MA (US)

(21) Appl. No.: 17/772,769

(22) PCT Filed: Oct. 28, 2020
(86) PCT No.: PCT/SG2020/050618

§ 371 Date: Apr. 28, 2022

(30) Foreign Application Priority Data
Oct. 28, 2019 (IT) 102019000019822

(51) Int. Cl. C12N 15/115; C12Q 1/6811; C12N 15/10

(52) U.S. Cl. CPC C12N 15/115; C12Q 1/6811; C12N 15/1048; C12N 2310/16; C12N 2310/322

(57) ABSTRACT

An aptamer, capable of inhibiting DNA methyltransferase 1 (DNMT1) for use in therapy of diseases characterised by aberrant DNA methylation, e.g. cancer. Method for identifying inhibitors of DNA methyltransferase. An aptamer, capable of inhibiting DNA methyltransferase 1 (DNMT1) for use in therapy of diseases characterised by aberrant DNA methylation, e.g. cancer. SELEX method for identifying aptamers of DNA methyltransferase optionally using 2-fluoro-pyrimindine nucleotide derivatives.

### TECHNICAL FIELD

The present invention relates to aptamer. In particular, the present invention relates to nucleic acid aptamer capable of inhibiting DNMT1.

### BACKGROUND

DNA methylation is a key epigenetic signature implicated in regulation of gene expression. Methylation of CpG-rich promoters is carried out by the members of the DNA methyltransferase (DNMT) family (DNMT1, DNMT2, DNMT3A, DNMT3B and DNMT3L). Numerous studies have established a link between aberrant promoter DNA methylation and cancer. Aberrant epigenetic modifications probably occur at an early stage of the tumor development and are reversible offering a unique possibility to alter gene function reprogramming malignant cellular transformation. In this view, epigenetic targeting by specific and effective inhibitors could lead to the development of clinically relevant strategies for cancer therapy.

Aptamers are small (6-30 kD) synthetic nucleic acids functioning as high affinity ligands. They are selected through a process known as SELEX (systematic evolution of ligands by exponential enrichment) that relies on the ability of the target protein to select high-affinity ligands from a random pool of nucleic acids. Besides being cost-effective and relatively easy to manipulate, aptamers demonstrate high affinity for their targets (in the low nanomolar range) and specificity similar to monoclonal antibodies and high tissue penetration comparable to small molecules. Moreover, aptamers are neither immunogenic nor toxic. All these features make them ideal candidate molecules for both diagnostic and clinical applications.

Indeed, the first aptamer-based drug for the treatment of macular degeneration was approved by the Food and Drug Administration (FDA) in 2004. Owing to their unique features, later studies in preclinical models and clinical trials have shown promising results for the use of aptamers as anti-cancer drug, to the extent they are considered the “new avatar” of personalized medicine for cancer patients.

Therefore, there is a need to provide alternative aptamer-based drug. There is a need to provide alternative aptamer-based drug for use in treating cancer in patients in need thereof.

### SUMMARY

In one aspect, there is provided an aptamer capable of inhibiting DNA methyltransferase 1 (DNMT1), wherein the aptamer comprises a stem loop structure deriving from DiR:ecCEBPA that is capable of interacting with DNMT1 to thereby inhibit DNMT1. In some embodiments, the aptamer is capable of reducing DNMT1 function by at least about 30%, and/or the aptamer is capable of increasing CEBPA (CCAAT/enhancer-binding protein alpha) levels, and/or the aptamer is capable of reducing the viability of a cancerous cell.

In some embodiments, the aptamer comprises a stem structure comprising two or more pairs of nucleotides, and/or the aptamer comprises a loop structure formed by four or more nucleotides.

In some embodiments, the aptamer is an RNA-aptamer.

In some embodiments, the aptamer comprises between 10 to 61 nucleotides, and/or the aptamer further comprises a modification capable of enhancing nuclease resistance, optionally the modification is a 2′-fluoro-, 2′-methoxy-, 2′-methoxyethyl-, and/or 2,-amino-modified nucleotides, optionally the aptamer comprises 2′-Fluoro-Pyrimidines (2′F-Py) modification.

In some embodiments, the aptamer comprises a nucleotide sequence that is at least 70% identical to any one of sequences shown in FIG. 6, optionally the aptamer comprises and/or consist of and/or has nucleotide sequence having at least 70% sequence identity to sequences selected from the group consisting of 5′ CUGAGCUCAUGGCGAGGCUUCU 3′ (SEQ ID NO: 9), 5′ UGGGCUGAGCUCAUGGCGAGGCUUC 3′ (SEQ ID NO: 67), 5′ CUGAGGCCUAACGAAGGCUUCU 3′ (SEQ ID NO: 68), 5′ CUGAGGCCUAACGAAGGCUUCU 3′ (SEQ ID NO: 68), 5′ CUGAGGUAAUGGCGAGGCUUCU 3′ (SEQ ID NO: 69), 5′ AGGUAAUGGCGAGGCUUCUUAUCUG 3′ (SEQ ID NO: 70), 5′ UUACUGGGCUGAGGUAAUGGCGAGG 3′ (SEQ ID NO: 71), and 5′ CTGAGGTAATGGCGAGGCTTCT 3′ (SEQ ID NO: 72).

In another aspect, there is provided an aptamer as disclosed for use in therapy.

In yet another aspect, there is provided a pharmaceutical composition comprising the aptamer as described herein.

In yet another aspect, there is provided a method of treating or preventing a disease characterized by aberrant DNA methylation in a subject in need thereof, the method comprising administering an effective amount of the aptamer as described herein or the pharmaceutical composition as described herein to modulate DNA methylation in the subject in need thereof.

In yet another aspect, there is provided a method of regulating DNA methylation in a subject in need thereof, the method comprising administering an effective amount of the aptamer as described herein or the pharmaceutical composition as described herein to modulate DNA methylation in the subject in need thereof.

In some embodiments, the method comprises administering the aptamer as described herein or the pharmaceutical composition as described herein to thereby inhibit DNMT1 activity in the subject in need thereof, optionally wherein the subject has a condition/disease characterized by changes in DNA methylation or aberrant DNA methylation that may include, but is not limited to, cancer, autoimmune diseases, genetic disorders, metabolic disorders, psychological disorders, and aging, optionally the disease characterized by aberrant DNA methylation is chronic myelogenous leukemia (CML), and/or non-small cell lung cancer (NSCLC), including adenocarcinoma (such as human alveolar basal epithelial adenocarcinoma) and squamous cell carcinoma.

In yet another aspect, there is provided a method of producing and/or selecting inhibitor(s) of a DNA methyltransferase, the method comprising: preparing one or more libraries of variants by introducing one or more alterations in an aptamer sequence capable of interacting with the DNA methyltransferase, wherein the one or more alterations is in a central region of the aptamer sequence and/or introduces a 2′-fluoro-pyrimidines modification; contacting/incubating the variants with a target DNA methyltransferase to allow the variants to bind to the target DNA methyltransferase; separating the variant(s) bound to the target DNA methyltransf erase from the unbound variant(s); and recovering the variant(s) bound to the target DNA methyltransferase to obtain the inhibitor(s) of the DNA methyltransferase.

In some embodiments, the preparing step further comprises attaching/adding a primer sequence to the variant, optionally the preparing step further comprises attaching/adding a promoter to the variant, optionally the variant comprises a stem and loop structure.

In some embodiments, wherein preparing the one or more libraries of variants comprises preparing sub-libraries of variants by introducing the one or more alterations in different pre-determined regions within the central region to form different sub-libraries of variants having alterations in different pre-determined regions.

In some embodiments, the variants comprise one or more flanking regions that are free of alteration.

In some embodiments, the method comprising mixing variants from each sub-library to form a diverse pool of variants for the contacting/incubating step.

In some embodiments, the method further comprising truncating the variants to obtain shortened variants retaining a stem and loop structure.

In some embodiments, wherein the DNA methyltransferase comprises DNMT1.

In some embodiments, wherein the one or more alterations is a randomization of the sequence and/or a 2′-fluoro-pyrimidines modification.

### BRIEF DESCRIPTION OF THE DRAWINGS

Exemplary embodiments of the invention will be better understood and readily apparent to one of ordinary skill in the art from the following written description, by way of example only, and in conjunction with the drawings, in which:

FIG. 1. Selection of DNMT1-specific aptamer by SELEX. a. Binding ability of R5 sequence modified with 2′F-Py (R5-F) on DNMT1 purified protein detected by ELONA assay; b. Stem—loop predicted structures of R5 and long R5 (R5L). The black arrows indicate R5 sequence within R5L; c. Site-specific randomized sub-libraries used as starting pool for the SELEX cycles; d. Scheme of the SELEX rounds. Each round include steps of: i) incubation of the RNA pool with glutathione-coupled magnetic beads (counter-selection); ii) recovering of unbound sequences; iii) incubation of the unbound sequences with purified GST-tagged DNMT1 protein (selection); iv) partitioning of the bound sequences with glutathione-coupled magnetic beads; v) recovering and amplification of bound sequences by RT-PCR.

FIG. 2. Analyses of individual DNMT1-specific aptamers selected by SELEX. a. Dendrogram of the individual sequences cloned after the SELEX rounds. The three sequences chosen for further analyses are shown in the box; b. Alignment of the central sequences of the three selected aptamers from SELEX (Ce-49; Ce-9 and Ce-10) and R5; c. Binding of biotinylated selected aptamers and R5-L with DNMT1 purified protein was analyzed by ELONA assay. Anti-DNMT1 antibody (mAb, Active Motif) was used as a positive control. Error bars depict mean±s.d.

FIG. 3. Optimization of DNMT1-specific aptamers. a. Predicted secondary structures of Ce-49, Ce-9 and Ce-10 and the designed short aptamers (indicated in the box). Two distinct folded structures and corresponding short aptamers were predicted for Ce-10 and indicated as Ce-10-1 and Ce-10-2, respectively. The black-arrows indicate the sequences of the short versions within the corresponding long aptamer. Ce-10-R5 was designed based on the central linear region of Ce-10; b. Binding of biotinylated short aptamers and R5-F on DNMT1 purified protein was analyzed by ELONA assay. Anti-DNMT1 antibody (mAb, Active Motif) were used as a positive control. Error bars depict mean±s.d.; c. Short aptamers and R5-F serum stability were measured in 85% human serum for indicated times. At each time point, RNA-serum samples were collected and evaluated by electrophoresis with 15% denaturing polyacrylamide gel. Gels were stained with ethidium bromide and the intensity of the bands quantified. Signal intensity was expressed relative to T0.

FIG. 4. Affinity and in vivo binding of DNMT1-specific aptamers. a. Analysis of Ce-49 sh and Ce-10 sh binding affinity to DNMT1 assessed by the Blitz system (ForteBIO). The background values obtained with mutR5 used as a negative control were subtracted from the values obtained with the aptamers; REMSA showing stronger Ce-49 sh and Ce-10 sh binding ability to DNMT1 than two alternative secondary structures of the original R5F (R5modified with 2′F-Py). Unrelated RNA and DNA are two positive controls. R01(unable to form stem-loop-like structures) is used as negative control; c. Increasing amount of Ce-49 sh and Ce-10 sh biotinylated aptamers were incubated in the presence or absence of HSA purified protein. No significant difference was detected between the blank control and the aptamers by ELONA. Error bars depict mean±s.d.; d. Aptamer-mediated pull-down. Protein extract from K562 cells were incubated with the biotinylated R5-F, Ce-49 sh and Ce-10-2 sh. Bound proteins were purified on streptavidin beads and immunoblotted with anti-DNMT1 antibodies.

FIG. 5. Functional activity of the aptamers. (a) Activity of purified DNMT1 protein was analyzed in vitro by DNMT1 inhibitor screening assay in the absence (“-”) or in the presence of indicated aptamers. Results are expressed as percentage relative to the activity of DNMT1 protein alone; b. DNMT1 inhibitor screening assay performed with K-562 nuclear extracts from untreated cells (NT), treated with a sequence unable to bind DNMT1 (control) or transfected with the respective aptamers Ce-10-2 sh and Ce-49 sh. Results are expressed as percentage relative to the activity detected in NT sample; c. expression levels 72 hours upon aptamers transfection; d, e. Cell viability of CML K562 cells (d) or NSCLC Calu-1 and A549 cells (e) transfected with indicated aptamers or Cont. for 72 hours. In (a-d) Error bars depict mean±s.d.; R5-F: R5 sequence modified with 2′F-Py.

FIG. 6. Sequence Alignment. Alignment of the individual sequences cloned after the SELEX rounds.

FIG. 7. Aptamer binding to HSA controls. For the binding to HSA assay, controls were performed by incubating aptamers at 200 nM with DNMT1 protein (left) or by using specific antibodies to check the effective coating of the plates (right).

FIG. 8. R5 and control binding to DNMT1. Bio-Layer Interferometry dose-response measurements of 2′F-pyrimidine modified R5 (DNMT1 bait) (a) or mut-R5 (b) (Cont., used as a negative control) binding to DNMT1 functionalized-biosensors. (c), Binding curves derived from BLItz analyses of DNMT1 bait. Curves were fitted with a 1:1 binding model using GraphPad Prism 6.

FIG. 9. Aptamer specificity. (a-c) Binding measured by bio-layer interferometry of Ce-49 sh (a), Ce-10-2 sh (b) and 2′F-pyrimidine modified R5 (DNMT1 bait) (c) to DNMT3A (left panels), DNMT3B (middle panels) and KAT5 protein (right panels) immobilized on separate biosensors. Aptamers were tested at the reported concentrations.

FIG. 10. Functional specificity of the best aptamers. Activity of purified DNMT3A (left) or B (right) proteins was analysed in vitro in the absence (-) or in the presence of indicated aptamers and expressed as percentage relative to the activity of DNMT protein alone. Bars depict mean±SD.

FIG. 11. DNA methylation analyses. (a) Heatmap of differentially methylated CpG regions (DMR) in K562 transfected with Ce-49 sh, Ce-10-2 sh or control (Cont.) aptamers. (b) Volcano plots reporting the significantly DMRs for Ce-49 sh (upper panel) and Ce-10-2 sh (lower panel). (c) DMRs overlapping between Ce-49 sh and Ce-10-2 sh. (d) Heatmap of top ranked Gene ontology terms for genes corresponding to the overlapping hypo-methylated CpGs shared by Ce-49 sh and Ce-10-2 sh.

### DETAILED DESCRIPTION

RNAs partnering with DNA methyltransferase 1 (DNMT1), termed DNMT1-interacting RNAs (DiRs), are epigenetic modulators shown to be capable of inhibiting the activity of the enzyme, thereby preventing methylation and silencing of the respective DiR-regulated genes. The RNA-DNMT1 association is widespread (with nearly 6000 gene loci involved) and negatively correlates with DNA methylation profiles. Therefore, DiRs have revealed a previously unknown mechanism of DNA-methylation-regulation by RNAs, raising the possibility that synthetic RNA molecules can be used to control DNA methylation. By combining the unique features of aptamers with the inherent properties of RNA to block DNA methylation, aptamers able to bind DNMT1 and interfere with its function have been successfully developed and are presented herein.

The presented disclosure therefore provides an RNA aptamer-based-platform to inhibit DNMT1. The herein described molecules represent a tool to correct aberrant methylation in cancer and other diseases characterized by aberrant DNA methylation.

Epigenetic targeting by specific and effective inhibitors could lead to the development of clinically relevant strategies for cancer therapy. So far, the two FDA approved compounds to inhibit DNMTs and reduce global DNA methylation are: 5-Azacytidine and 5-Aza 2′ deoxy-cytidine. However, the lack of selectivity, toxicity and chemical instability represent serious concerns for the use of these nucleoside analogues. Therefore, there is a need to develop smart and safe epigenetic drugs able to modulate the expression of a small set of genes.

Aptamers are emerging as therapeutic tools in treating diseases. An FDA approved aptamer drug against vascular endothelial growth factor (VEGF), Pegaptanib (Macugen; Pfizer/Eyetech), had shown clinical effectiveness in treating age-related macular degeneration. Furthermore, clot-buster NU172 (ARCA Biopharma) is now in phase II clinical trial to reduce clotting during coronary artery bypass graft surgery. In cancer therapy, although the use of aptamer is in still at its early stage, the outcome had been promising. The high affinity aptamer AS1411 causing self-destruction in several renal, lung and breast cancer cell lines, has shown to have anti-tumour effect and it is currently in a phase II clinical trial for the treatment of leukaemia.

In this disclosure, the focus is on a major epigenetic player, DMNT1, which governs the genomic methylation landscape in cancer cells. The approach described herein is the first-ever attempt in targeting epigenetic complexes using aptamers and it provides a novel strategy to modulate DNA methylation with many advantages such as low immunogenicity, small size, high batch fidelity, easy production, increased chemical stability and versatility.

The present disclosure provides DNMT1 inhibition using RNA-aptamers. Aptamers of the present disclosure are molecules highly stable in vivo, with a greater affinity and specificity to their targets, low toxicity, and no immunogenicity.

In one aspect, the present invention provides an aptamer capable of inhibiting DNA methyltransferase 1 (DNMT1), wherein the aptamer comprises a stem loop structure deriving from DiR:ecCEBPA that is capable of interacting with DNMT1 to thereby inhibit DNMT1. In some examples, the aptamer comprises a stem loop structure deriving from DiR:ecCEBPA that is capable of interacting at high affinity (KD value <100 nM) interacting with DNMT1 to thereby inhibit DNMT1. Also disclosed is a polynucleotide/an oligonucleotide capable of inhibiting DNA methyltransferase 1 (DNMT1).

In some embodiments, the aptamer is capable of reducing DNMT1 function by at least about 30%, and/or the aptamer is capable of increasing CEBPA (CCAAT/enhancer-binding protein alpha) levels, and/or the aptamer is capable of reducing the viability of a cancerous cell. In some examples, the polynucleotide/oligonucleotide is capable of reducing DNMT1 function by at least about 30%. In some examples, the polynucleotide/oligonucleotide is capable of reducing DNMT1 function by at least about 35% to 100%, or at least about 35%, or at least about 40%, or at least about 45%, or at least about 50%, or at least about 55%, or at least about 60%, or at least about 65%, or at least about 70%, or at least about 75%, or at least about 80%, or at least about 85%, or at least about 90%, or at least about 95%, or at least about 100% in in vitro and/or in vivo.

In some examples, the polynucleotide/oligonucleotide has a binding affinity of between 50 nM to 90 nM, or at least 50 nM, or at least 60 nM, or at least 70 nM, or at least 80 nM, or not more than 100 nM, or not more than 90 nM, or not more than 80 nM, or not more than 70 nM, or not more than 60 nM, or not more than 50 nM, or not more than 40 nM, or not more than 30 nM, or not more than 20 nM, or not more than 10 nM.

In some examples, the polynucleotide/oligonucleotide is capable of increasing CEBPA (CCAAT/enhancer-binding protein alpha) levels.

In some examples, the polynucleotide/oligonucleotide is capable of reducing the viability of a cancerous cell. In some examples, the cell may include, but is not limited to any cancerous cells such as a chronic myelogenous leukemia cell (CML cell) and/or non-small cell lung cancer including adenocarcinoma (such as human alveolar basal epithelial cells A549) and squamous cell carcinoma (such as Calu-1). In some examples, the cell is a K562 CML cell and/or non-small cell lung cancer (NSCLC) Calu-1 and/or A549 cells.

In some examples, the polynucleotide/oligonucleotide is an aptamer.

As used herein, the term “aptamer” refers to a polynucleotide/oligonucleotide that binds specifically and/or binds with high affinity to a target molecule. Under defined conditions, aptamers may fold into a specific two-dimensional and/or three-dimensional structure. As described herein, the aptamers of the present disclosure interact specifically and with high affinity with DNMT1 to thereby prevent methylation and silencing the respective DNMT1-regulated genes.

The aptamer as disclosed herein comprises or consists of a sequence of nucleic acid molecules, the nucleotides. In some examples, the aptamer of the present disclosure consists of a nucleotide sequence as defined herein (for examples with reference to the Experimental Section, Figures and Sequences disclosed herein).

The aptamer as described herein may comprise unmodified and/or modified D- and/or L-nucleotides. According to the common one letter code of nucleic acid bases “C” or stands for cytosine, “A” or stands for adenine, “G” or stands for guanine, and “T” or stands for thymine if the nucleotide sequence is a DNA sequence and “T” or stands for a uracil nucleotide if the nucleotide sequence is a RNA sequence. Unless specified, the term “nucleotide” may refer to ribonucleotides and deoxyribonucleotides.

The aptamer as described herein may comprise or consist of a DNA- or an RNA-nucleotide sequence and, thus, may be referred to as DNA-aptamer or RNA-aptamer, respectively. It is understood that, if the aptamer of the invention comprises an RNA-nucleotide sequence, within the sequence motifs specified throughout the present invention “T” stands for uracil.

Whilst the present disclosure may refer to RNA-aptamers or RNA-nucleotide sequences, it is understood that the respective DNA-aptamers or DNA-nucleotide sequences are also comprised in the present disclosure. In some examples, the aptamer is capable of forming a stem-loop structure. Without wishing to be bound by theory, it is believed that the stem-loop structure allows the polynucleotide/oligonucleotide/aptamer to be capable of binding/interacting with DNMT1. At the same time, the stem-loop structure also confers stability to the folding of the polynucleotide/oligonucleotide/aptamer.

In some examples, the aptamer comprises a stem loop structure deriving from the DiR-ecCEBPA, which is capable of interacting with DNMT1 to thereby inhibit the enzyme. In some examples, the polynucleotide/oligonucleotide/aptamer or portion thereof comprises a stem loop structure that is substantially similar or identical to a stem loop structure derived from DiR-ecCEBPA. As would be evident in the Experimental Section and further aspects of the present disclosure, the polynucleotide/oligonucleotide/aptamer or portion thereof, e.g. stem-loop structure, may be selected from DiR:ecCEBPA by the SELEX approach.

The polynucleotides/oligonucleotides/aptamers as described herein or portions thereof may assume a secondary structure similar to that assumed by DNA when interacting with DNMT1. Without wishing to be bound by theory, it is believed that the binding dynamics of stem loop structures may change based on the stability of the structures and the likelihood of them being formed under physiological conditions. In some examples, the stem loop binding of the aptamers/polynucleotides/oligonucleotides to DNMT1 is unique. In some examples, the aptamer comprises a stem structure comprising two or more pairs of nucleotides. In some examples, the aptamer may comprise a stem structure comprising 2, or 3, or 4, or 5, or 6, or 7, or 8, or more pairs of nucleotides. It is believed that the stem portion of the aptamer may have any suitable stem length. In some examples, the aptamer comprises a loop structure formed by sterically sufficient number of nucleobases. As such, in some examples, the aptamer may comprise a loop structure formed by four or more nucleobases. In some examples, the aptamer may comprise a loop structure formed by 4, or 5, or 6, or 7, or 8, or 9, or 10, or 11, or 12, or 13, or 14, or 15 nucleotides. In some examples, the aptamer may comprise a loop structure formed by about 4 to 8 nucleobases.

In some examples, the aptamer is an RNA-aptamer.

In some examples, the aptamer comprises between 10 to 61 nucleotides. In some examples, the aptamer comprises between 10 to 60 nucleotides, or 22 nucleotides, or 23 nucleotides, or 24 nucleotides, or 25 nucleotides. In some examples, the aptamer may comprise or consist of about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 nucleotides. In some examples, the aptamer may comprise or consist of 22 nucleotides or 23 nucleotides, or 24 nucleotides, or 25 nucleotides. In some examples, the aptamer may be of between 10 to 61 mer.

In some examples, the aptamer further comprises a modification capable of enhancing nuclease resistance. In some examples, at least one polynucleotide aptamer may be modified to prevent nuclease degradation. In some examples, at least one polynucleotide aptamer may be modified to increase the circulating half-life of the aptamer after administration to a subject. In some examples, the nucleotides of the aptamers may be linked by phosphate linkages. In some examples, one or more of the internucleotide linkages may be modified linkages, e.g., linkages that are resistant to nuclease degradation. The term “modified internucleotide linkage” includes all modified internucleotide linkages known in the art or that come to be known and that, from reading this disclosure, one skilled in the art will conclude is useful in connection with the presently disclosed methods. internucleotide linkages may have associated counterions, and the term is meant to include such counterions and any coordination complexes that can form at the internucleotide linkages. Modifications of internucleotide linkages include, without limitation, phosphorothioates, phosphorodithioates, methylphosphonates, 5′-alkylenephosphonates, 5′-methylphosphonate, 3′-alkylene phosphonates, borontrifluoridates, borano phosphate esters and selenophosphates of 3′-5′ linkage or 2′-5′ linkage, phosphotriesters, thionoalkylphosphotriesters, hydrogen phosphonate linkages, alkyl phosphonates, alkylphosphonothioates, arylphosphonothioates, phosphoroselenoates, phosphorodiselenoates, phosphinates, phosphoramidates, 3′-alkylphosphoramidates, aminoalkylphosphoramidates, thionophosphoramidates, phosphoropiperazidates, phosphoroanilothioates, phosphoroanilidates, ketones, sulfones, sulfonamides, carbonates, carbamates, methylenehydrazos, methylenedimethylhydrazos, formacetals, thioformacetals, oximes, methyleneiminos, methylenemethyliminos, thioamidates, linkages with riboacetyl groups, aminoethyl glycine, silyl or siloxane linkages, alkyl or cycloalkyl linkages with or without heteroatoms of, for example, 1 to 10 carbons that can be saturated or unsaturated and/or substituted and/or contain heteroatoms, linkages with morpholino structures, amides, polyamides wherein the bases can be attached to the aza nitrogens of the backbone directly or indirectly, and combinations of such modified intemucleotide linkages.

In some examples, the modification is a 2′-fluoro-, 2′-methoxy-, 2′-methoxyethyl-, and/or 2,-amino-modified nucleotides. In some examples, the aptamers may comprise a nucleotide sequence containing 2′-modified nucleotides, e.g. 2′-fluoro-, 2′-methoxy-, 2′-methoxyethyl- and/or 2,-amino-modified nucleotides. The aptamer may also comprise a mixture of deoxyribonucleotides, modified deoxyribonucleotides, ribonucleotides and/or modified ribonucleotides. Respectively, the terms “2′-fluoro-modified nucleotide”, “2′-methoxy-modified nucleotide”, “2′-methoxyethyl-modified nucleotide” and/or “2-amino-modified nucleotide” refer to modified ribonucleotides and modified deoxyribonucleotides. In some examples, the aptamer may comprise a 2′-Fluoro-Pyrimidines (2′F-Py) modification. In some examples, the aptamer comprises 2′-Fluoro-Pyrimidines (2′F-Py) modification.

The aptamer may comprise further modifications. Such modifications encompass e.g. alkylation, i.e. methylation, arylation or acetylation of at least one nucleotide, the inclusion of enantiomers and/or the fusion of aptamers with one or more other nucleotides or nucleic acid sequences. Such modifications may comprise e.g. 5′- and/or 3′-PEG- or 5′- and/or 3′-CAP-modifications. Alternatively or in addition, the aptamer may comprise modified nucleotides, such as, but is not limited to, locked-nucleic acids, 2′-fluoro-, 2′-methoxy- and/or 2′-amino-modified nucleotides.

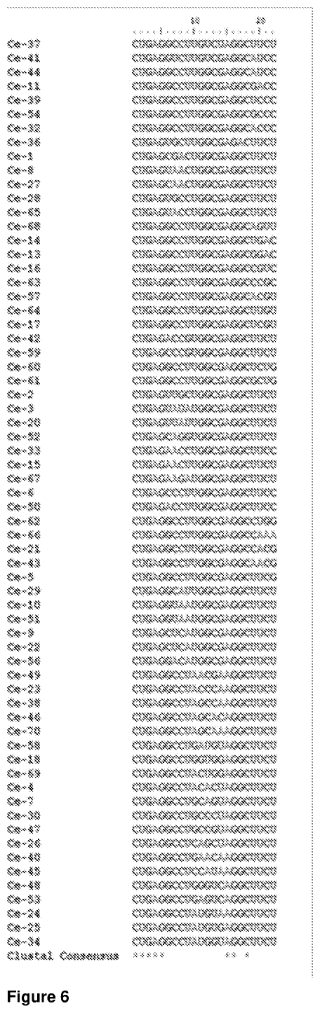

In some examples, the aptamer comprises a nucleotide sequence that is at least 70% identical to any one of sequences shown in FIG. 6, or any one or more of the following sequences:

>Ce-1
(SEQ ID NO: 1)
CUGAGCGACUGGCGAGGCUUCU;
>Ce-2
(SEQ ID NO: 2)
CUGAGUUGCUGGCGAGGCUUCU;
>Ce-3
(SEQ ID NO: 3)
CUGAGUAUAUGGCGAGGCUUCU;
>Ce-4
(SEQ ID NO: 4)
CUGAGGCCUACACUAGGCUUCU;
>Ce-5
(SEQ ID NO: 5)
CUGAGGCCUUGGCGAGGCUUCG;
>Ce-6
(SEQ ID NO: 6)
CUGAGCCCUUGGCGAGGCUUCC;
>Ce-7
(SEQ ID NO: 7)
CUGAGGCCUGCAGUAGGCUUCU;
>Ce-8
(SEQ ID NO: 8)
CUGAGUAACUGGCGAGGCUUCU;
>Ce-9
(SEQ ID NO: 9)
CUGAGCUCAUGGCGAGGCUUCU;
>Ce-10
(SEQ ID NO: 10)
CUGAGGUAAUGGCGAGGCUUCU;
>Ce-11
(SEQ ID NO: 11)
CUGAGGCCUUGGCGAGGCGACC;
>Ce-13
(SEQ ID NO: 12)
CUGAGGCCUUGGCGAGGCGGAC;
>Ce-14
(SEQ ID NO: 13)
CUGAGGCCUUGGCGAGGCUGAC;
>Ce-15
(SEQ ID NO: 14)
CUGAGAACUUGGCGAGGCUUCU;
>Ce-16
(SEQ ID NO: 15)
CUGAGGCCUUGGCGAGGCCGUC;
>Ce-17
(SEQ ID NO: 16)
CUGAGGCCUUGGCGAGGCUCGU;
>Ce-18
(SEQ ID NO: 17)
CUGAGGCCUGGUGGAGGCUUCU;
>Ce-20
(SEQ ID NO: 18)
CUGAGUUAUUGGCGAGGCUUCU;
>Ce-21
(SEQ ID NO: 19)
CUGAGGCCUUGGCGAGGCCACG;
>Ce-22
(SEQ ID NO: 20)
CUGAGCUCAUGGCGAGGCUUCU;
>Ce-23
(SEQ ID NO: 21)
CUGAGGCCUACCCAAGGCUUCU;
>Ce-24
(SEQ ID NO: 22)
CUGAGGCCUAUGUAAGGCUUCU;
>Ce-25
(SEQ ID NO: 23)
CUGAGGCCUAUGUGAGGCUUCU;
>Ce-26
(SEQ ID NO: 24)
CUGAGGCCUCAGCUAGGCUUCU;
>Ce-27
(SEQ ID NO: 25)
CUGAGCAACUGGCGAGGCUUCU;
>Ce-28
(SEQ ID NO: 26)
CUGAGUGCCUGGCGAGGCUUCU;
>Ce-29
(SEQ ID NO: 27)
CUGAGGCAUUGGCGAGGCUUCU;
>Ce-30
(SEQ ID NO: 28)
CUGAGGCCUGCCCUAGGCUUCU;
>Ce-32
(SEQ ID NO: 29)
CUGAGGCCUUGGCGAGGCACCC;
>Ce-33
(SEQ ID NO: 30)
CUGAGAACCUGGCGAGGCUUCC;
>Ce-34
(SEQ ID NO: 31)
CUGAGGCCUAUGGUAGGCUUCU;
>Ce-36
(SEQ ID NO: 32)
CUGAGUGCUUGGCGAGACUUCU;
>Ce-37
(SEQ ID NO: 33)
CUGAGGCCUUGUCUAGGCUUCU;
>Ce-38
(SEQ ID NO: 34)
CUGAGGCCUAGCCAAGGCUUCU;
>Ce-39
(SEQ ID NO: 35)
CUGAGGCCUUGGCGAGGCUCCC;
>Ce-40
(SEQ ID NO: 36)
CUGAGGCCUGAACAAGGCUUCU;
>Ce-41
(SEQ ID NO: 37)
CUGAGGUCUUGUCGAGGCAUCC;
>Ce-42
(SEQ ID NO: 38)
CUGAGACCGUGGCGAGGCUUCU;
>Ce-43
(SEQ ID NO: 39)
CUGAGGCCUUGGCGAGGCAACG;
>Ce-44
(SEQ ID NO: 40)
CUGAGGCCUUGGCGAGGCAUCC;
>Ce-45
(SEQ ID NO: 41)
CUGAGGCCUCCAUAAGGCUUCU;
>Ce-46
(SEQ ID NO: 42)
CUGAGGCCUAGCACAGGCUUCU;
>Ce-47
(SEQ ID NO: 43)
CUGAGGCCUGCCGUAGGCUUCU;
>Ce-48
(SEQ ID NO: 44)
CUGAGGCCUGGGUCAGGCUUCU;
>Ce-49
(SEQ ID NO: 45)
CUGAGGCCUAACGAAGGCUUCU;
>Ce-50
(SEQ ID NO: 46)
CUGAGACCUUGGCGAGGCUUCC;
>Ce-51
(SEQ ID NO: 47)
CUGAGGUAAUGGCGAGGCUUCU;
>Ce-52
(SEQ ID NO: 48)
CUGAGCAGGUGGCGAGGCUUCU;
>Ce-53
(SEQ ID NO: 49)
CUGAGGCCUGAGUCAGGCUUCU;
>Ce-54
(SEQ ID NO: 50)
CUGAGGCCUUGGCGAGGCGCCC;
>Ce-56
(SEQ ID NO: 51)
CUGAGGACAUGGCGAGGCUUCU;
>Ce-57
(SEQ ID NO: 52)
CUGAGGCCUUGGCGAGGCACGU;
>Ce-58
(SEQ ID NO: 53)
CUGAGGCCUGAUGUAGGCUUCU;
>Ce-59
(SEQ ID NO: 54)
CUGAGCCCGUGGCGAGGCUUCU;
>Ce-60
(SEQ ID NO: 55)
CUGAGGCCUUGGCGAGGCUCUG;
>Ce-61
(SEQ ID NO: 56)
CUGAGGCCUUGGCGAGGCGCUG;
>Ce-62
(SEQ ID NO: 57)
CUGAGGCCUUGGCGAGGCCUGG;
>Ce-63
(SEQ ID NO: 58)
CUGAGGCCUUGGCGAGGCCCGC;
>Ce-64
(SEQ ID NO: 59)
CUGAGGCCUUGGCGAGGCUUGU;
>Ce-65
(SEQ ID NO: 60)
CUGAGUACCUGGCGAGGCUUCU;
>Ce-66
(SEQ ID NO: 61)
CUGAGGCCUUGGCGAGGCCAAA;
>Ce-67
(SEQ ID NO: 62)
CUGAGAAGAUGGCGAGGCUUCU;
>Ce-68
(SEQ ID NO: 63)
CUGAGGCCUUGGCGAGGCAGUU;
>Ce-69
(SEQ ID NO: 64)
CUGAGGCCUACUGGAGGCUUCU;
>Ce-70
(SEQ ID NO: 65)
CUGAGGCCUAGCAAAGGCUUCU.

In some examples, at least one aptamer comprises a nucleotide sequence that is at least 70% identical, e.g., at least 75%, 80%, 85%, 90%, 91%, 92%, 93%, 94%, 95%, 96%, 97%, 98%, or 99% identical to any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, and 65. In some embodiments, the aptamer consists of a nucleotide sequence that is at least 70% identical, e.g., at least 75%, 80%, 85%, 90%, 91%, 92%, 93%, 94%, 95%, 96%, 97%, 98%, or 99% identical to any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, and 65. In some embodiments, the aptamer comprises a nucleotide sequence that is identical to a fragment of any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, and 65 of at least 5 contiguous nucleotides, e.g., at least about 10, 15, 20, or 25 contiguous nucleotides. In some embodiments, the aptamer comprises a nucleotide sequence that is at least 70% identical, e.g., at least 75%, 80%, 85%, 90%, 91%, 92%, 93%, 94%, 95%; 96%, 97%, 98%, or 99% identical to a fragment of any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, and 65 of at least 5 contiguous nucleotides, e.g., at least about 10, 15, 20, or 25 contiguous nucleotides.

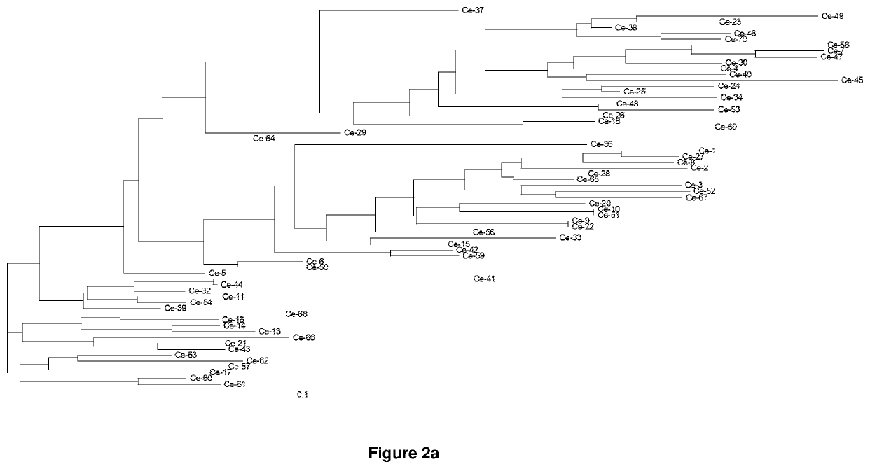

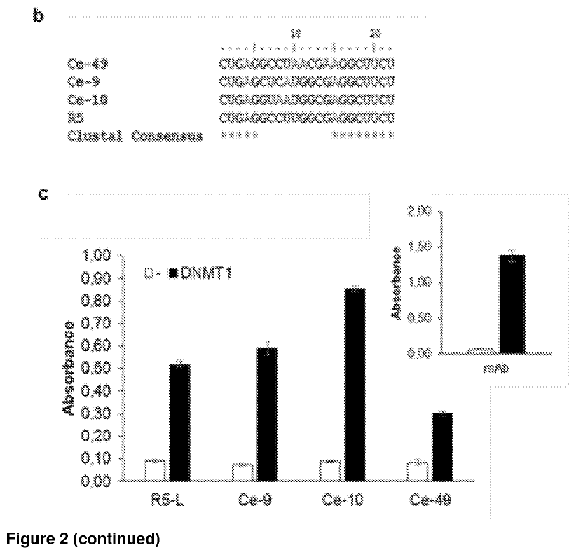

In some examples, the aptamers may comprise 1, or 2, or 3, or 4, or 5 or more mismatches as compared to the stem loop RNA structure R5 as disclosed in FIG. 2b or CUGAGGCCUUGGCGAGGCUUCU (R5; SEQ ID NO: 66). In some examples, the aptamers may comprise 1, or 2, or 3, or 4, or 5 or more point-mutation as compared to the stem loop RNA structure R5 as disclosed in FIG. 2b or CUGAGGCCUUGGCGAGGCUUCU (R5; SEQ ID NO: 66). In some examples, the aptamers may comprise one or more mismatches and/or one or more point-mutations as compared to the stem loop RNA structure R5 as disclosed in FIG. 2b or CUGAGGCCUUGGCGAGGCUUCU (R5; SEQ ID NO: 66).

In some examples, the aptamers described herein comprise both ribonucleotides and deoxyribonucleotides. In some embodiments, the aptamers described herein comprise and/or consist of ribonucleotides.

In some examples, the fragments and/or analogs of the aptamers of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, and 66 have a substantially similar activity as one or more of the aptamers of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, and 66.

“Substantially similar,” as used herein, refers to specific binding to DNMT1, and in some examples also refers to an inhibitory activity on the aptamers in blocking DNA methylation by specific binding with DNMT1, that is at least about 20% of the inhibitory activity of one or more of the aptamers of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, and 66.

When a number of individual, distinct aptamer sequences for a single target molecule have been obtained and sequenced as described herein, the sequences can be examined for “consensus sequences.” As used herein, “consensus sequence” refers to a nucleotide sequence or region (which might or might not be made up of contiguous nucleotides) that is found in one or more regions of at least two aptamers, the presence of which can be correlated with aptamer-to-target-binding or with aptamer structure.

A consensus sequence can be as short as three nucleotides long. It also can be made up of one or more noncontiguous sequences with nucleotide sequences or polymers of hundreds of bases long interspersed between the consensus sequences. Consensus sequences can be identified by sequence comparisons between individual aptamer species, which comparisons can be aided by computer programs and other tools for modeling secondary and tertiary structure from sequence information. Generally, the consensus sequence will contain at least about 5 to 20 nucleotides. An exemplary consensus sequence or clustal consensus may be observed in FIG. 6.

The term “fragment” refers to a nucleotide sequence of reduced length relative to the reference nucleic acid and comprising, over the common portion, a nucleotide sequence identical to the reference nucleic acid. Such a nucleic acid fragment according to the presently disclosed subject matter may be, where appropriate, included in a larger polynucleotide of which it is a constituent. Such fragments comprise, or alternatively consist of, oligonucleotides ranging in length from at least 6, 8, 9, 10, 12, 15, 18, 20, 21, 22, 23, 24, 25, 30, 39, 40, 42, 45, 48, 50, 51, 54, 57, 60, 63, 66, 70, 75, 78, 80, 90, 100, 105, 120, 135, 150, 200, 300, 500, 720, 900, 1000 or 1500 consecutive nucleotides of a nucleic acid according to the presently disclosed subject matter.

The term “percent identity,” as known in the art, is a relationship between two or more polypeptide sequences or two or more polynucleotide sequences, as determined by comparing the sequences. In the art, “identity” also means the degree of sequence relatedness between polypeptide or polynucleotide sequences, as the case may be, as determined by the match between strings of such sequences. “Identity” and “similarity” can be readily calculated by known methods.

In some examples, the aptamer may comprises and/or consist of and/or may have nucleotide sequence having at least 70% sequence identity to sequences selected from the group consisting of 5′ CUGAGCUCAUGGCGAGGCUUCU 3′ (i.e. Ce-9; SEQ ID NO: 9 or SEQ ID NO: 20), 5′ UGGGCUGAGCUCAUGGCGAGGCUUC 3′ (i.e. Ce-9 sh; SEQ ID NO: 67), 5′ CUGAGGUAAUGGCGAGGCUUCU 3′ (i.e. Ce-10; SEQ ID NO: 69), 5′ CUGAGGCCUAACGAAGGCUUCU 3′ (i.e. Ce-49 sh; SEQ ID NO: 68), 5′ CUGAGGCCUAACGAAGGCUUCU 3′ (i.e. Ce-49; SEQ ID NO; 68), 5′ AGGUAAUGGCGAGGCUUCUUAUCUG 3′ (i.e. Ce-10-1 sh; SEQ ID NO: 70), 5′ UUACUGGGCUGAGGUAAUGGCGAGG 3′ (i.e. Ce-10-2 sh; SEQ ID NO: 71), and 5′ CTGAGGTAATGGCGAGGCTTCT 3′ (i.e. Ce-10 R5; SEQ ID NO: 72).

Once an aptamer sequence, according to the presently disclosed subject matter, is identified, the aptamer may by synthesized by any method known to those of skill in the art. In some embodiments, aptamers may be produced by chemical synthesis of oligonucleotides and/or ligation of shorter oligonucleotides. In some embodiments, polynucleotides may be used to express the aptamers, e.g., by in vitro transcription, polymerase chain reaction amplification, or cellular expression. The polynucleotides may be DNA and/or RNA and may be single-stranded or double-stranded. In some embodiments, the polynucleotide is a vector which may be used to express the aptamer. The vector may be, e.g., a plasmid vector or a viral vector and may be suited for use in any type of cell, such as mammalian, insect, plant, fungal, or bacterial cells. The vector may comprise one or more regulatory elements necessary for expressing the aptamers, e.g., a promoter, enhancer, transcription control elements, etc.

Several methods known in the art may be used to propagate a polynucleotide according to the presently disclosed subject matter. Once a suitable host system and growth conditions are established, recombinant expression vectors can be propagated and prepared in quantity. As described herein, the expression vectors which can be used include, but are not limited to, the following vectors or their derivatives: human or animal viruses such as vaccinia virus or adenovirus; insect viruses such as baculovirus; yeast vectors; bacteriophage vectors (e.g., lambda), and plasmid and cosmid DNA vectors, to name but a few.

A “vector” is any means for the cloning of and/or transfer of a nucleic acid into a host cell. A vector may be a replicon to which another DNA segment may be attached so as to bring about the replication of the attached segment. A “replicon” is any genetic element (e.g., plasmid, phage, cosmid, chromosome, virus) that functions as an autonomous unit of DNA replication in vivo, i.e., capable of replication under its own control. The term “vector” includes both viral and nonviral means for introducing the nucleic acid into a cell in vitro, ex vivo or in vivo. A large number of vectors known in the art may be used to manipulate nucleic acids, incorporate response elements and promoters into genes, etc. Possible vectors include, for example, plasmids or modified viruses including, for example bacteriophages such as lambda derivatives, or plasmids.

The term “transfection” means the uptake of exogenous or heterologous RNA or DNA by a cell. A cell has been “transfected” by exogenous or heterologous RNA or DNA when such RNA or DNA has been introduced inside the cell. A cell has been “transformed” by exogenous or heterologous RNA or DNA when the transfected RNA or DNA effects a phenotypic change. The transforming RNA or DNA can be integrated (covalently linked) into chromosomal DNA making up the genome of the cell.

In some examples, the aptamers may be linked to conjugates that increase the circulating half-life, e.g., by decreasing nuclease degradation or renal filtration of the aptamer. Conjugates may include, for example, amino acids, peptides, polypeptides, proteins, antibodies, antigens, toxins, hormones, lipids, nucleotides, nucleosides, sugars, carbohydrates, polymers such as polyethylene glycol and polypropylene glycol, as well as analogs or derivatives of all of these classes of substances. Additional examples of conjugates also include steroids, such as cholesterol, phospholipids, di- and tri-acylglycerols, fatty acids, hydrocarbons that may or may not contain unsaturation or substitutions, enzyme substrates, biotin, digoxigenin, and polysaccharides. Further examples include thioethers such as hexyl-S-tritylthiol, thiocholesterol, acyl chains such as dodecandiol or undecyl groups, phospholipids such as di-hexadecyl-rac-glycerol, triethylammonium 1,2-di-O-hexadecyl-rac-glycero-3-H-phosphonate, polyamines, polyethylene glycol, adamantane acetic acid, palmityl moieties, octadecylamine moieties, hexylaminocarbonyl-oxycholesterol, famesyl, geranyl and geranylgeranyl moieties, such as polyethylene glycol, cholesterol, lipids, or fatty acids. Conjugates can also be detectable labels. For example, conjugates can be fluorophores. Conjugates can include fluorophores such as TAMRA, BODIPY, cyanine derivatives such as Cy3 or Cy5 Dabsyl, or any other suitable fluorophore known in the art. A conjugate may be attached to any position on the terminal nucleotide that is convenient and that does not substantially interfere with the desired activity of the aptamer that bears it, for example the 3′ or 5′ position of a ribosyl sugar. A conjugate substantially interferes with the desired activity of an aptamer if it adversely affects its functionality such that the ability of the aptamer to bind DNMT1 is reduced by greater than 80% in a binding assay.

In some examples, the aptamers as described herein that specifically bind DNMT1 may be linked to conjugates capable of mediating delivery into a cell of interest. “Cell of interest” as used herein refers to cells with aberrant DNA methylation, such cells in a patient suspected of having or developing a proliferative disease (for example cancer). Such conjugates that mediate intracellular delivery of the aptamers as described herein that specifically bind a cell of interest include other aptamers that are known to specifically enter cells of interest (referred to herein as “delivery aptamers”) or other ligands that bind receptors on a cell of interest and are internalized by the cell. Such conjugates may further include detectable labels such as fluorophores to facilitate methods of screening cells of interest containing the aptamers as described herein that specifically bind DNMT1.

Where the conjugates are delivery aptamers, the delivery aptamers and the aptamers as described herein that specifically bind DNMT1 may be linked, for example, covalently or functionally through nucleic acid duplex formation. At least one of the linked aptamers may be partly or wholly comprised of 2′-modified RNA or DNA such as 2′F, 2′OH, 2′OMe, 2′allyl, 2′MOE (methoxy-O-methyl) substituted nucleotides and may contain polyethylene glycol (PEG)-spacers and abasic residues. Covalent linkages for delivery aptamers and other ligands may include, for example, a linking moiety such as a nucleic acid moiety, a PNA moiety, a peptidic moiety, a disulfide bond or a polyethylene glycol (PEG) moiety. In some examples, at least one polynucleotide aptamer comprises at least one 2′-fluoro nucleotide.

In another aspect, there is provided a polynucleotide and/or an oligonucleotide and/or an aptamer and/or an RNA aptamer as described herein for use in therapy and/or as a medicine and/or for cancer therapy.

In yet another aspect, there is provided a pharmaceutical composition comprising the polynucleotide and/or an oligonucleotide and/or an aptamer and/or an RNA aptamer as described herein.

In yet another aspect, there is provided a use of a polynucleotide and/or an oligonucleotide and/or an aptamer and/or an RNA aptamer as described herein in the manufacture of a medicament for regulating DNA methylation and/or treating a disease characterized by aberrant DNA methylation.

In yet another aspect, there is provided method of regulating DNA methylation in a subject in need thereof, the method comprising administering an effective amount of the aptamer as disclosed herein or the pharmaceutical composition as disclosed herein inhibiting DNA methyltransferase 1 (DNMT1) activity to modulate DNA methylation in the subject in need thereof. Also disclosed is a method of regulating DNA methylation on a subject, the method comprises inhibiting DNA methyltransferase 1 (DNMT1) activity to modulate DNA methylation. In some examples, the subject may be a cell population in the lab and/or a subject patient such as a human patient.

In yet another aspect, there is provided a method of treating or preventing a disease characterized by aberrant DNA methylation in a subject in need thereof, the method comprising administering an effective amount of the aptamer as disclosed herein or the pharmaceutical composition as disclosed herein inhibiting DNA methyltransferase 1 (DNMT1) activity to modulate DNA methylation in the subject in need thereof. Also disclosed is a method of treating or preventing a disease characterized by aberrant DNA methylation in a subject in need thereof, the method comprises inhibiting DNA methyltransferase 1 (DNMT1) activity to modulate DNA methylation in the subject in need thereof.

As used herein, the terms “treat,” treating,” “treatment,” and the like, are meant to decrease, suppress, attenuate, diminish, arrest, the underlying cause of a disease, disorder, or condition, or to stabilize the development or progression of a disease, disorder, condition, and/or symptoms associated therewith. The terms “treat,” “treating,” “treatment,” and the like, as used herein can refer to curative therapy, prophylactic therapy, and preventative therapy. Accordingly, as used herein, “treating” means either slowing, stopping or reversing the progression of aberrant or undesired DNA methylation, including reversing the progression to the point of eliminating the presence of aberrant or undesired DNA methylation and/or reducing or eliminating the amount of aberrant or undesired DNA methylation, or the amelioration of symptoms associated with aberrant or undesired DNA methylation. The treatment, administration, or therapy can be consecutive or intermittent. Consecutive treatment, administration, or therapy refers to treatment on at least a daily basis without interruption in treatment by one or more days.

As used herein, conditions/diseases characterized by changes in DNA methylation or aberrant DNA methylation may include, but is not limited to, aging, aberrant proliferative diseases such as cancer, autoimmune disease, genetic disorders, metabolic disorders, psychological disorders, and the like.

The term “cancer” encompasses a disease involving both pre-malignant and malignant cancer cells. In some examples, cancer refers to a localized overgrowth of cells that has not spread to other parts of a subject, i.e., a benign tumor. In other examples, cancer may be referring to a malignant tumor, which has invaded and destroyed neighboring body structures and spread to distant sites.

In some example, the disease characterized by aberrant DNA methylation may include, but is not limited to, acute lymphoblastic leukemia, acute myeloid leukemia, adrenocortical carcinoma, AIDS-related cancer, AIDS-related lymphoma, anal cancer, appendix cancer, astrocytomas (childhood cerebellar or cerebral), basal cell carcinoma, bile duct cancer, bladder cancer, bone cancer, brainstem glioma, brain tumors (cerebellar astrocytoma, cerebral astrocytoma/malignant glioma, ependymoma, medulloblastoma, supratentorial primitive neuroectodermal tumors, visual pathway and hypothalamic gliomas), breast cancer, bronchial adenomas/carcinoids, Burkitt lymphoma, carcinoid tumors (childhood, gastrointestinal), carcinoma of unknown primary, central nervous system lymphoma (primary), cerebellar astrocytoma, cerebral astrocytoma/malignant glioma, cervical cancer, childhood cancers, chronic lymphocytic leukemia, chronic myelogenous leukemia, chronic myeloproliferative disorders, colon cancer, cutaneous T-cell lymphoma, desmoplastic small round cell tumor, endometrial cancer, ependymoma, esophageal cancer, Ewing's sarcoma in the Ewing family of tumors, extracranial germ cell tumor (childhood), extragonadal germ cell tumor, extrahepatic bile duct cancer, eye cancers (intraocular melanoma, retinoblastoma), gall bladder cancer, gastric (stomach) cancer, gastrointestinal carcinoid tumor, gastrointestinal stromal tumor, germ cell tumors (childhood extracranial, extragonadal, ovarian), gestational trophoblastic tumor, gliomas (adult, childhood brain stem, childhood cerebral astrocytoma, childhood visual pathway and hypothalamic), gastric carcinoid, hairy cell leukemia, head and neck cancer, hepatocellular (liver) cancer, Hodgkin lymphoma, hypopharyngeal cancer, hypothalamic and visual pathway glioma (childhood), intraocular melanoma, islet cell carcinoma, Kaposi sarcoma, kidney cancer (renal cell cancer), laryngeal cancer, leukemias (acute lymphoblastic, acute myeloid, chronic lymphocytic, chronic myelogenous, hairy cell), lip and oral cavity cancer, liver cancer (primary), lung cancers (non-small cell, small cell), lymphomas (AIDS-related, Burkitt, cutaneous T-cell, Hodgkin, non-Hodgkin, primary central nervous system), macroglobulinemia (Waldenstrom), malignant fibrous histiocytoma of bone/osteosarcoma, medulloblastoma (childhood), melanoma, intraocular melanoma, Merkel cell carcinoma, mesotheliomas (adult malignant, childhood), metastatic squamous neck cancer with occult primary, mouth cancer, multiple endocrine neoplasia syndrome (childhood), multiple myeloma/plasma cell neoplasm, mycosis fungoides, myelodysplastic syndromes, myelodysplastic/myeloproliferative diseases, myelogenous leukemia (chronic/chronic myelogenous leukemia (CML), myeloid leukemias (adult acute, childhood acute), multiple myeloma, myeloproliferative disorders (chronic), nasal cavity and paranasal sinus cancer, nasopharyngeal carcinoma, neuroblastoma, non-Hodgkin lymphoma, non-small cell lung cancer, oral cancer, oropharyngeal cancer, osteosarcoma/malignant fibrous histiocytoma of bone, ovarian cancer, ovarian epithelial cancer (surface epithelial-stromal tumor), ovarian germ cell tumor, ovarian low malignant potential tumor, pancreatic cancer, pancreatic cancer (islet cell), paranasal sinus and nasal cavity cancer, parathyroid cancer, penile cancer, pharyngeal cancer, pheochromocytoma, pineal astrocytoma, pineal germinoma, pineoblastoma and supratentorial primitive neuroectodermal tumors (childhood), pituitary adenoma, plasma cell neoplasia, pleuropulmonary blastoma, primary central nervous system lymphoma, prostate cancer, rectal cancer, renal cell carcinoma (kidney cancer), renal pelvis and ureter transitional cell cancer, retinoblastoma, rhabdomyosarcoma (childhood), salivary gland cancer, sarcoma (Ewing family of tumors, Kaposi, soft tissue, uterine), Sezary syndrome, skin cancers (nonmelanoma, melanoma), skin carcinoma (Merkel cell), small cell lung cancer, small intestine cancer, soft tissue sarcoma, squamous cell carcinoma, squamous neck cancer with occult primary (metastatic), stomach cancer, supratentorial primitive neuroectodermal tumor (childhood), T Cell lymphoma (cutaneous), testicular cancer, throat cancer, thymoma (childhood), thymoma and thymic carcinoma, thyroid cancer, thyroid cancer (childhood), transitional cell cancer of the renal pelvis and ureter, trophoblastic tumor (gestational), unknown primary site (adult, childhood), ureter and renal pelvis transitional cell cancer, urethral cancer, uterine cancer (endometrial), uterine sarcoma, vaginal cancer, visual pathway and hypothalamic glioma (childhood), vulvar cancer, Waldenstrom macroglobulinemia, and Wilms tumor (childhood).

In some examples, the disease characterized by aberrant DNA methylation may be cancer. In some examples, the cancer may be chronic myelogenous leukemia (CML) and/or non-small cell lung cancer (NSCLC) including adenocarcinoma (such as human alveolar basal epithelial adenocarcinoma), squamous cell carcinoma, and/or its combination thereof.

In some examples, the method comprises administering the polynucleotide/oligonucleotide of any of the preceding AS to thereby inhibit DNMT1 activity in the subject in need thereof.

In yet another aspect, there is provided a method of producing and/or selecting inhibitor(s) of a DNA methyltransferase, the method comprising: preparing one or more libraries of variants by introducing one or more alterations in an aptamer sequence capable of interacting with the DNA methyltransferase, wherein the one or more alterations in the aptamer sequence comprises introducing a 2′-fluoro-pyrimidines modification; contacting/incubating the variants with a target DNA methyltransferase to allow the variants to bind to the target DNA methyltransferase; separating the variant(s) bound to the target DNA methyltransferase from the unbound variant(s); and recovering the variant(s) bound to the target DNA methyltransferase to obtain the inhibitor(s) of the DNA methyltransferase.

Thus, disclosed herein is a method of producing/selecting high affinity and specificity inhibitor(s) of a DNA methyltransferase by evolving existing interacting RNAs. The method allows evolving short RNA aptamers with the introduction of conformational constraints and randomized regions. In some examples, the method may comprise the preparation of one or more libraries of variants (different libraries of variants (for example, there may be one, or two, or three, or four different libraries of variants)) by introducing one or more alterations in different positions of an aptamer sequence capable of interacting with the DNA methyltransferase; contacting/incubating the variants with a target DNA methyltransferase to allow the variants to bind to the target DNA methyltransferase; separating the variant(s) bound to the target DNA methyltransferase from the unbound variant(s); and recovering the variant(s) bound to the target DNA methyltransferase to obtain the inhibitor(s) of the DNA methyltransferase. In some examples, the preparation of the library of variants further comprises introducing a 2′-fluoro-pyrimidines modification. Also disclosed is a method of producing/selecting inhibitor(s) of a DNA methyltransferase, the method comprising:

preparing one or more libraries of variants by introducing one or more alterations in a polynucleotide/oligonucleotide sequence capable of interacting with the DNA methyltransferase;

contacting/incubating the one or more libraries of variants with a target DNA methyltransferase to allow the variants to bind to the target DNA methyltransferase;

separating the variant(s) bound to the target DNA methyltransferase from the unbound variant(s); and

recovering the variant(s) bound to the target DNA methyltransferase to obtain the inhibitor(s) of the DNA methyltransferase.

In some examples, the preparing step further comprises introducing modification to the variants, e.g. a 2′-fluoro-pyrimidines modification, to enhance nuclease resistance.

In some examples, the preparing step further comprises introducing modification to the variants to enhance nuclease resistance. In some examples, the modification is introduced to at the 2′ position of the nucleotides. In various embodiments, the modification comprises a 2′-fluoro-, 2′-methoxy-, 2′-methoxyethyl- and/or 2,-amino-modified modification. In some examples, the modification is introduced to the pyrimidines in the variants. In one example, the modification comprises a 2′-fluoro-pyrimidines modification. In some examples, the modification does not substantially alter the binding affinity of the variants for the DNA methyltransferase.

In various examples, the modified variants show good serum stability. In various examples, the modified variants are substantially stable in human serum for at least about 24 hours, at least about 36 hours, at least about 48 hours, at least about 60 hours or at least about 72 hours. In various examples, the modified variants are no more than about 40%, no more than about 35%, no more than about 30%, no more than about 25%, no more than about 20%, no more than about 15% or no more than about 10% degraded when incubated in human serum for 72 hours at 37° C.

In some examples, the preparing step further comprises attaching/adding a primer sequence to the variant.

In some examples, a primer sequence is attached/added to the 5′ end of the variants. In some examples, a primer sequence is attached/added to the 3′ end of the variants. In some examples, a primer sequence is attached/added to each of the 5′ and 3′ ends of the variants. In some examples, the primer sequence attached/added to each of the 5′ and 3′ ends of the variants are constant primer sequences. In some examples, the primer sequence facilitates PCR amplification and/or transcription.

In some examples, the primers may include, but not limited to, 5′-TAATACGACTCACTATAGGGCTGAAGGGGTTACTGGG-3′ (forward with T7 RNA polymerase promoter; SEQ ID NO: 73), 5′-CTCCTCCCCGGGGCAGATA-3′ (reverse; SEQ ID NO: 74), and the like.

In various examples, the primer sequence comprises and/or consists of and/or has nucleotide sequence having at least about 70% sequence identity, at least about 75% sequence identity, at least about 80% sequence identity, at least about 85% sequence identity, at least about 90% sequence identity, at least about 95% sequence identity, at least about 96% sequence identity, at least about 97% sequence identity, at least about 98% sequence identity, at least about 99% sequence identity, or at least about 100% sequence identity with the sequences selected from the group consisting of 5′ TAATACGACTCACTATAGGGCTGAAGGGGTTACTGGG-3′ (SEQ ID NO: 73) and 5′-CTCCTCCCCGGGGCAGATA-3′ (SEQ ID NO: 74).

In some examples, the preparing step further comprises attaching/adding a promoter to the variant. In one example, a T-7 promoter is attached/added to the variant. In various embodiments, the promoter facilitates in vivo transcription.

In some examples, the variant comprises a stem and loop structure.

In some examples, the polynucleotide/oligonucleotide sequence comprises a stem and loop structure. In some examples, the variant comprises a stem and loop structure. In some examples, the inhibitor comprises a stem and loop structure. In some examples, the inhibitor or the variant modified from the polynucleotide/oligonucleotide sequence retains the stem and loop structure of the polynucleotide/oligonucleotide sequence. In some examples, wherein the introducing one or more alterations in the polynucleotide/oligonucleotide sequence comprises introducing one or more alterations in a central region of the polynucleotide/oligonucleotide sequence.

In some examples, the central region is flanked by a 5′ portion and a 3′ portion of the polynucleotide/oligonucleotide sequence. In some examples, the 5′ portion and/or the 3′ portion comprises or has a length of at least about 5 nucleotides, at least about 10 nucleotides, or at least about 15 nucleotides. In some examples, the 5′ portion and/or the 3′ portion comprises or has a length of from about 10 to about 30 nucleotides, from about 15 to about 25 nucleotides, or from about 17 to about 22 nucleotides. In some examples, the 5′ portion and/or the 3′ portion comprises or has a length of about 15 nucleotides, about 16 nucleotides, about 17 nucleotides, about 18 nucleotides, about 19 nucleotides, about 20 nucleotides, about 21 nucleotides, about 22 nucleotides, about 23 nucleotides, about 24 nucleotides or about 25 nucleotides. In some examples, the flanking regions are fixed regions i.e. the sequences in the flanking regions are not altered. As illustrated in the Examples section, the SELEX as described herein uses as starting pool R5 variants with different fully randomized short regions. The SELEX library was designed starting from a construct containing R5 variants as central region flanked by two constant 20- and 19-nt long naïve regions (flanking the R5 sequence within the ecCEBPA) at the 5′ and 3′ ends, respectively, as fixed regions. Importantly, the library components should retain stem-and-loop-like structure required for the DNMT1-RNA interaction. Different libraries (three or more) may be produced by randomizing different R5 regions. Therefore, in some embodiments, the central region is randomized and/or altered and the flanking regions are fixed and not randomized or free of randomization. In some examples, the flanking region may comprise native DiR: ecCEBPA sequence (or complementary sequence thereof).

As used here, the term “altered” refers to a modification of one or more nucleic acid within a sequence. For example, the alteration may comprise the introduction of a modification in an aptamer (such as modification with 2′ fluoro pyrimidines). At the same time, the term “randomized” refers to the possible modifications of one or more nucleotide within a sequence (4ⁿ total modification considering as “n” the number of randomized nucleotides). Therefore, term “randomized” may refer to the unpredictable or unsystematic manner a part of the polynucleotide/oligonucleotide sequence as described herein is synthesized. In addition, as exemplified in the Experimental section, in some examples, the term “altered” may also encompass the term “randomized”. Therefore, an “alteration” as disclosed herein may refer to a modification and/or a randomization of a sequence. For example, in some examples, when one or more alterations in a central region of a nucleic acid sequence is introduced, the alteration may be a randomization of the sequence of the central region of the nucleic acid sequence. In some examples, the randomized sequence of the central region of the nucleic acid sequence may further comprise a modification of the nucleic acid (such as a modification with 2′ fluoro pyrimidines). In some examples, the one or more alterations is a randomization of the sequence and/or a 2′fluoro-pyrimidines modification. In some examples, the method comprises preparing one or more library of variants comprising an aptamer having a randomized central region whilst retaining a stem-and-loop-like structure that interacts with the DNMT1. In some examples, the method further comprises altering the one or more variants with a modification (such as 2′fluoro-pyrimidines modification).

In some examples, introducing one or more alterations in a central region of the polynucleotide/oligonucleotide sequence comprises degenerating the central region of the polynucleotide/oligonucleotide sequence or a portion thereof.

In some examples, preparing a library of variants comprises preparing sub-libraries of variants by introducing the one or more alterations in different pre-determined regions within the central region to form different sub-libraries of variants having alterations in different pre-determined regions.

In some examples, the number of different pre-determined regions is at least about two, at least about three, at least about four or at least about five. In one example, where the central region has three different pre-determined regions R1, R2, and R3, the method comprises introducing one or more alterations in the R1 region (but not the R2 and R3 regions) to form a sub-library SL1 of variants, introducing one or more alterations in the R2 region (but not the R1 and R3 regions) to form a sub-library SL2 of variants, and comprises introducing one or more alterations in the R3 region (but not the R1 and R2 regions) to form a sub-library SL3 of variants. Thus, sub-library SL1 contains only variants having alteration(s) in the R1 region, sub-library SL2 contains only variants having alteration(s) in the R2 region and sub-library SL3 contains only variants having alteration(s) in the R3 region. This also means that, in this example, if the R1 region in one variant is altered, the R2 and R3 regions of that variant are not altered. If the R2 region in one variant is altered the R1 and R3 regions of that variant are not altered. If the R3 region in one variant is altered the R1 and R2 regions of that variant are not altered. In some examples therefore, in each variant, the one or more alterations is introduced in no more than one of at least about two different pre-determined regions, at least about three different pre-determined regions, at least about four different pre-determined regions, or at least about five different pre-determined regions.

In some examples, each of the different pre-determined regions are non-overlapping.

In some examples, some or all of the pre-determined regions form a continuous sequence. In other words, some or all of the pre-determined regions may be directly adjacent to each other. In some embodiments, some or all of the pre-determined regions do not form a continuous sequence. In other words, some or all of the pre-determined regions may not be directly adjacent to each other i.e. some or all of the pre-determined regions are separated from each other by at least one nucleotide disposed between them. In one example, where alterations is introduced in each of three non-pre-determined regions R1, R2 and R3, at least two of the three pre-determined regions (e.g. R1 and R2) form a continuous sequence while the remaining pre-determined region (e.g. R3) is located separately from the two pre-determined regions.

In some examples, the pre-determined region comprises or has a length of no more than about 10 nucleotides, no more than about 9 nucleotides, no more than about 8 nucleotides, no more than about 7 nucleotides, no more than about 6 nucleotides or no more than about 5 nucleotides. In various embodiments, the pre-determined region comprises or has a length of from about 2 to about 7 nucleotides, or from about 3 to about 6 nucleotides. In various embodiments, the pre-determined region comprises or has a length of about 2 nucleotides, about 3 nucleotides, about 4 nucleotides, about 5 nucleotides, about 6 nucleotides or about 7 nucleotides.

In some examples, the method comprising mixing variants from each sub-library to form a diverse pool of variants for the contacting/incubating step.

In some examples, the variants from each sub-library are mixed in equimolar ratio. For example, if there are 3 sub-libraries SL1, SL2 and SL3, the variants from each sub-library SL1, SL2 and SL3 may be mixed in the ratio 1:1:1.

In some examples, the target DNA methyltransferase comprises a tagged DNA methyltransferase.

In one example, the target DNA methyltransferase is tagged with glutathione S-Transferase (GST). In one example, the GST is tagged to the N-terminal of the target DNA methyltransferase. In one example, the method further comprises counter selecting for variants that bind to glutathione-coupled magnetic beads prior to the contacting/incubating step. The variants that bind to the glutathione-coupled magnetic beads may be removed by a magnetic separator before the remaining variants are contacted/incubated with the GST-tagged target DNA methyltransferase. This is to avoid the non-specific variants from being recovered in tandem with variants that show affinity for the DNA methyltransferase when separation is subsequently carried out using glutathione-coupled magnetic beads. Thus, in various examples, the method further comprises a counter selection step to remove non-specific variants.

In various examples, at the contacting/incubating, the ratio of the amount of variants/pool of variants to the amount of the target DNA methyltransferase is at least about 20:1, at least about 25:1 or at least about 30:1. In various examples, the ratio of the amount of variants/pool of variants to the amount of the target DNA methyltransferase is from about 20:1 to about 40:1. In various examples, the ratio of the amount of variants/pool of variants to the amount of the target DNA methyltransferase is about 20:1, 25:1, 30:1, 35:1 or 40:1. In one example the ratio of the amount of variants/pool of variants to the amount of the target DNA methyltransferase is 30:1 pmol.

In some examples, the recovered variants are subjected to at least one more round of contacting/incubating, separating and recovering as described hereinabove to select for inhibitor(s) having high affinity for the DNA methyltransferase.

In some examples, the recovered variants are subjected to at least about one, at least about two, at least about three or least about four more rounds of contact/incubation, separation and recovery as described hereinabove to select for variant(s)/inhibitor(s) having high affinity for the DNA methyltransferase. In some examples, in the about two, about three, about four or about five rounds of contact/incubation, separation and recovery, an increasing number of washes is used to progressively improve the stringency of the selection and enhance the recovering of variant(s)/inhibitor(s) with high affinity for the DNA methyltransferase. In various embodiments, in each round of contact/incubation, separation and recovery, the variants are washed at least about one time, at least about two times, at least about four times, at least about five times, about least about six times, at least about seven times, at least about eight times, about least about nine times or at least about ten times. In one example, the variants are washed two times in a first round of contact/incubation, separation and recovery, three times in a second round and four times in a third round.

In some examples, the method further comprises amplifying the recovered variants, optionally by polymerase chain reaction (PCR), further optionally by reverse transcriptase polymerase chain reaction (RT-PCR).

In various examples, the recovered variants are amplified before being subjected to one or more further rounds of contact/incubation, separation and recovery. In some examples, an error-prone PCR is used to introduce further mutation(s) in the amplicons before they are being subjected to one or more further rounds of contact/incubation, separation and recovery.

In various examples, the method comprises using a systematic evolution of ligands by exponential enrichment (SELEX) technique for the production/selection of inhibitor(s) of a DNA methyltransferase.

In various examples, the method further comprises cloning the recovered variants. In one example, the recovered variants are cloned through TA cloning system. In various examples, the recovered or cloned variants are isolated and sequenced.

In some examples, the method further comprising truncating the variants to obtain shortened variants retaining a stem and loop structure.

In some examples, the DNA methytransferase comprises DNMT1.

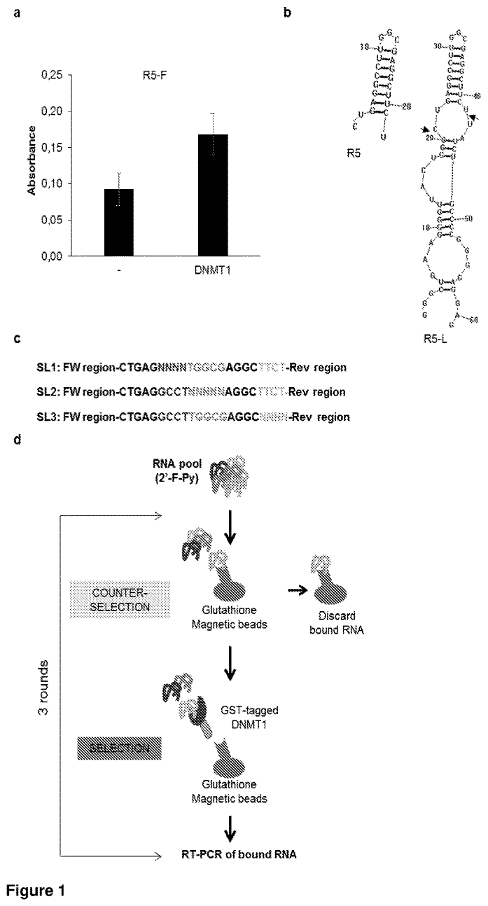

In some example, the method as described herein is exemplified in FIG. 1.

Additionally, when describing some embodiments, the disclosure may have disclosed a method and/or process as a particular sequence of steps. However, unless otherwise required, it will be appreciated the method or process should not be limited to the particular sequence of steps disclosed. Other sequences of steps may be possible. The particular order of the steps disclosed herein should not be construed as undue limitations. Unless otherwise required, a method and/or process disclosed herein should not be limited to the steps being carried out in the order written. The sequence of steps may be varied and still remain within the scope of the disclosure.

Further, in the description herein, the word “substantially” whenever used is understood to include, but not restricted to, “entirely” or “completely” and the like. In addition, terms such as “comprising”, “comprise”, and the like whenever used, are intended to be non-restricting descriptive language in that they broadly include elements/components recited after such terms, in addition to other components not explicitly recited. For an example, when “comprising” is used, reference to a “one” feature is also intended to be a reference to “at least one” of that feature. Terms such as “consisting”, “consist”, and the like, may, in the appropriate context, be considered as a subset of terms such as “comprising”, “comprise”, and the like. Therefore, in embodiments disclosed herein using the terms such as “comprising”, “comprise”, and the like, it will be appreciated that these embodiments provide teaching for corresponding embodiments using terms such as “consisting”, “consist”, and the like. Further, terms such as “about”, “approximately” and the like whenever used, typically means a reasonable variation, for example a variation of +/−5% of the disclosed value, or a variance of 4% of the disclosed value, or a variance of 3% of the disclosed value, a variance of 2% of the disclosed value or a variance of 1% of the disclosed value.

Furthermore, in the description herein, certain values may be disclosed in a range. The values showing the end points of a range are intended to illustrate a preferred range. Whenever a range has been described, it is intended that the range covers and teaches all possible sub-ranges as well as individual numerical values within that range. That is, the end points of a range should not be interpreted as inflexible limitations. For example, a description of a range of 1% to 5% is intended to have specifically disclosed sub-ranges 1% to 2%, 1% to 3%, 1% to 4%, 2% to 3% etc., as well as individually, values within that range such as 1%, 2%, 3%, 4% and 5%. The intention of the above specific disclosure is applicable to any depth/breadth of a range.

It will be appreciated by a person skilled in the art that other variations and/or modifications may be made to the specific embodiments without departing from the scope of the invention as broadly described. For example, in the description herein, features of different exemplary embodiments may be mixed, combined, interchanged, incorporated, adopted, modified, included etc. or the like across different exemplary embodiments. The present embodiments are, therefore, to be considered in all respects to be illustrative and not restrictive.

### EXPERIMENTAL SECTION

### Methods

### Library Preparation and In Vitro Transcription

Randomized sub-libraries were purchased from Genomics and PCR amplified by using 0.05 U/μl Fire Pol DNA Polymerase (Microtech) in a mix containing: 0,4 μM primers, 0.2 mM dNTPs. After 3 minutes initial denaturation at 95° C., the protocol used was: 10 cycles of: 95° C. for 30 seconds, 64° C. for 1 minute, 72° C. for 30 seconds; following final extension of 5 minutes at 72° C. Primers used were: forward (with T7 RNA Polymerase promoter): 5′ TAATACGACTCACTATAGGGCTGAAGGGGTTACTGGG-3′ (SEQ ID NO: 73); reverse: 5′-CTCCTCCCCGGGGCAGATA-3′ (SEQ ID NO: 74).

Equal amounts of each sub-libraries were mixed and transcribed. In vitro transcription was performed in the presence of 1 mM 2′-F pyrimidines using a mutant form of T7 RNA polymerase (Y639F). DNA template was incubated at 37° C. over night in a transcription mix containing: transcription buffer 1× (Epicentre Biotechnologies), 1 mM 2′F-Py (2′F-2′-dCTP and 2′F-2′-dUTP, TriLink Biotech, San Diego, Calif.), 1 mM ATP, 1 mM GTP (Thermo scientific), 10 mM dithiothreitol (DTT) (Thermo scientific), 0.5 u/μl RNAse inhibitors (Roche), 5 μg/ml inorganic pyrophosphatase (Roche), and 1.5 u/μl of the mutant T7 RNA polymerase (T7 R&DNA polymerase, Epicentre Biotechnologies). After transcription, any leftover DNAs were removed by DNase I (Roche) digestion and RNAs were purified by phenol:chloroform extraction, ethanol precipitation and gel purification on a denaturing 8% acrylamide/7 M Urea gel.

In vitro transcription in the presence of a mixture of 5′-biotin-G-monophosphate (TriLink Biotechnologies) and GTP (molar ratio 3:2) were performed for the biotinylation of the long aptamers.

SELEX procedure

For the SELEX strategy, GST-tagged DNMT1 was used as target for the selection. Recombinant Human DNMT1 with an N-terminal GST tag was purchased from Active Motif and Pierce™ Glutathione Magnetic Beads (Thermo Scientific) was used to separate aptamer-GST-tagged protein complexes.

Before each cycle of SELEX, 2′F-Py RNA pool was dissolved in RNAse free water and subjected to denaturation/renaturation steps of 85° C. for 5 min, ice for 2 min and 37° C. for 10 min. The RNA—protein incubation was performed in Binding Buffer (BB: 5 mM TrisHCl pH 7,5; 5 mM MgCl2; 1 mM DTT; 100 mM NaCl). At each cycle, the pool was first incubated for 30 min with Glutathione Magnetic Beads with a gentle rotation, as counter-selection step, and then the unbound RNA was recovered on a magnetic separator and used for selection. The recovered sequences were incubated with GST-tagged DNMT1 at room temperature for 30 min with a gentle rotation. Aptamer-protein complexes were purified on magnetic beads. The unbound was removed on a magnetic separator and the beads containing RNA-protein complexes were washed. Bound RNAs were recovered by TriFast (Euroclone) extraction and RT-PCR and finally transcribed for the following round.

### RT-PCR

RNA recovered at each SELEX round was reverse transcribed by using M-MuLV enzyme reverse Transcriptase (Roche) in a mix containing a specific buffer 5× (50 mM Tris-HCl pH 8.3, 40 mM KCl, 6 mM MgCl2, 10 mM DTT), 0,8 μM of Reverse primer, 1 mM dNTPs. The protocol used for the reaction was: 30 min at 42° C. and 30 min at 50° C. The obtained product was then amplified by error prone PCR reaction in the presence of high MgCl2 (7.5 mM) and dNTPs (1 mM).

### Cloning, Sequencing Bioinformatics Analysis

The final pool from SELEX was amplified by PCR, including in the program a 15 minutes final extension at 72° C. to

introduce A-overhangs. Individual sequences were cloned with TOPO-TA Cloning Kit (Invitrogen) according to the manufacturer' instruction. Single white clones were grown and DNA was extracted with plasmid Miniprep kit (Quiagen) and sequenced by Eurofins Genomics. Single aptamer sequences obtained were analyzed by using Multiple Sequence Alignment alignment tool (ClustalW2). Aptamer secondary structures were calculated by using RNAstructure Fold algorithms.

### Synthetic Oligonucleotides

Short RNA aptamers were purchased from Trilink
Biotechnologies
Ce-9 sh:
(SEQ ID NO: 67)
5’ UGGGCUGAGCUCAUGGCGAGGCUUC 3’
Ce-49 sh:
(SEQ ID NO: 68)
5’ CUGAGGCCUAACGAAGGCUUCU 3’
Ce-10-1 sh:
(SEQ ID NO: 70)
5’ AGGUAAUGGCGAGGCUUCUUAUCUG 3’
Ce-10-2 sh:
(SEQ ID NO: 71)
5’ UUACUGGGCUGAGGUAAUGGCGAGG 3’
Ce-10 R5:
(SEQ ID NO: 72)
5’ CTGAGGTAATGGCGAGGCTTCT 3’

### ELONA Assay

Microtiter High Binding plate (Nunc MaxiSorp) wells were coated with 30 nM of His-tagged DNMT1 (Active Motif) or HSA overnight at 4° C. All subsequent steps were performed at room temperature. After incubation, the plate was washed once with PBS and then blocked each well with 300 μl 3% BSA (AppliChem) in PBS for 2 hours. After two washes, the plate was incubated with 100 μL of 3′-biotinylated aptamers dissolved in PBS for 2 hours. Next, the plate was washed three times and streptavidin-conjugated horseradish peroxidase (HRP) (Sigma Aldrich) was added (1:10000 dilution). After 1-hour incubation, the plate was washed four times and the 3,3′,5,5′-Tetramethylbenzidine (TMB) substrate solution was added. The reaction was stopped with sulfuric acid (H₂SO₄) 0,16 M, forming a yellow reaction product that was read with microplate Reader (Thermo Scientific) at 450 nm.

### Human Serum Stability Assay

Oligonucleotides were incubated 4 μM in 85% human serum from T0 to 72 hours. Type AB Human Serum provided by Sigma Aldrich was used. At each time point 4 μl (16 pmol RNA) were recovered and incubated for 1 hours at 37° C. with 5 μl of proteinase K solution (20 mg/ml) in order to remove serum proteins that interfere with electrophoretic migration. Following proteinase K treatment, 18 μl of dye RNA (95% formamide, 10 mM EDTA, Bromophenol Blue, H₂O) was added to samples that were then stored at −80° C. All time point samples were separated by electrophoresis into 15% acrylamide/7 M Urea gel. The gel was stained with ethidium bromide and visualized by UV exposure.

### Cells and Transfection

CML K562 and NSCLC A549 cells were grown in RPMI medium supplemented with 10% FBS (Sigma). Transfections were performed using serum-free Opti-MEM and Lipofectamine 2000 reagent (Life technologies, Milan Italy) according to the manufacturer's protocol. Cells were transfected with 100 nmol/l of RNAs previously subjected to denaturation/renaturation steps.

### Aptamer-Mediated Pull Down

K562 cells were lysed with 10 mmol/l Tris-HCl pH 7.5 containing 200 nmol/l NaCl, 5 mmol/1 ethylendiaminetetraacetate (EDTA), 0.1% Triton X-100 and protease inhibitors. Extracts (500 μg in 0.5 ml lysis buffer) were incubated for 30 min with 200 nM heat denatured biotinylated aptamers with rotation. Following three washings with PBS cells, aptamer-protein complexes were purified on streptavidin beads (Thermo Fischer Scientific) for 2 hours. Beads were washed three times with PBS and bound proteins were recovered by adding Laemmli buffer and then analyzed by immunoblotting with anti-DNMT1 antibody (Active Motif).

### RT-qPCR

To analyze gene mRNA level, 1 mg of total RNA was reverse transcribed with iScript cDNA Synthesis Kit and amplified in real-time PCR with IQ-SYBR Green supermix (Bio-Rad, Hercules, Calif., USA). ΔΔCt method was used for relative mRNA quantization by applying the equation 2^{-ΔΔct.}

### Cell Viability Assay

Cells were seeded in 96-well plates (3×10³ cells/well) and were transfected with indicated sequences (100 nmol/l). Following 72 hours, cell viability was assessed by CellTiter 96 Proliferation Assay (Promega).

### Results

### Generation of Anti-DMNT1 Aptamers

The inventors of the present disclosure have previously demonstrated that a stem loop RNA structure of 22 nucleotides (R5) stemming the DiR: ecCEBPA is sufficient for the interaction with DNMT1 (Di Ruscio et al. Nature 2013). In order to evolve oligonucleotides with high affinity, specificity and stability, the inventors have developed a new protein SELEX (Systematic Evolution of Ligands by EXponential enrichment) strategy by randomizing R5 sequence and introducing 2′-Fluoro-Pyrimidines (2′F-Py) modifications to enhance the nuclease resistance. The modification of R5 with 2′F-Py (R5-F) does not alter its binding to DNMT1 as assessed by direct-Enzyme-Linked Oligonucleotides Assay (ELONA) assay (FIG. 1a).

A typical SELEX library contains a random central region flanked by two constant primer sequences required for PCR amplification and transcription. To design the library for the present disclosure, R5 sequence was used as central region to be randomized and the 5′ and 3′ portions of 20 and 19 nucleotides, respectively, flanking the R5 sequence within ecCEBPA, as fixed regions. T-7 promoter was then added to allow in vitro transcription. The resulting long version of R5 (indicated as R5-L) preserves R5 stem and loop structure (FIG. 1b).

To obtain a library of variants for the selection strategy, the degeneration of R5 sequence was proceeded. The inventors of the present disclosure carried out a site-specific randomization by chemical synthesis of three different R5 regions (4-5 bases each) generating three separate sub-libraries (SL1, SL2 and SL3) (FIG. 1c). The three sub-libraries were mixed at equimolar concentration and used as template for the transcription of the 2′F-Py modified RNA starting mixed pool. The pool was subjected to three protein-based SELEX cycles in which the Glutathione S-Transferase (GST)-tagged purified human recombinant full-length DNMT1 protein was used as target. As outlined in FIG. 1d, at each round, the pool was first incubated on glutathione-coupled magnetic beads for the counter selection step. The bound sequences were separated with a magnetic separator and the unbound aptamers were used for the selection on GST-tagged DNMT1 protein. Following the selection step, bound sequences were partitioned on glutathione-coupled magnetic beads and recovered by RT-PCR. During the three rounds, an increasing number of final washes was used to progressively improve the stringency of the selection and enhance the recovering of aptamers with high affinity for the target (Table 1).

TABLE 1
SELEX conditions

| | | | | | Number of |
|---|---|---|---|---|---|
| | RNA pool | Protein | RNA:protein | Number of | counter- |
| Round | (pmol) | (pmol) | ratio | washes | selection |
| 1 | 300 | 10 | 30:1 | 2 | 1 |
| 2 | 150 | 5 | 30:1 | 3 | 1 |
| 3 | 150 | 5 | 30:1 | 4 | 1 |

### Analyses of Individual Sequences

After the third SELEX round, the final enriched pool was cloned through TA cloning system and about seventy clones were isolated and sequenced. Sequences were aligned by Muscle Algorithm and clustered into families. Among the analyzed clones, variants coming from the three SLs were equally represented, indicating that there was not a preferential selection of specific region of mutation (FIG. 6).

The present disclosure identified three clusters of sequences with two aptamers (Ce-9 and Ce-10) representing the most enriched ones (FIG. 2a). These two aptamers present two mismatches among them and both had three mutation points compared to R5 (FIG. 2b). These sequences, together with Ce-49 that comes from a different cluster, were tested for the binding to DNMT1 purified protein by ELONA at 200 nM concentration. As shown in FIG. 2c, at this concentration, a good binding ability was detected for all the analyzed sequences as compared to R5-L.

### Sequence Optimization

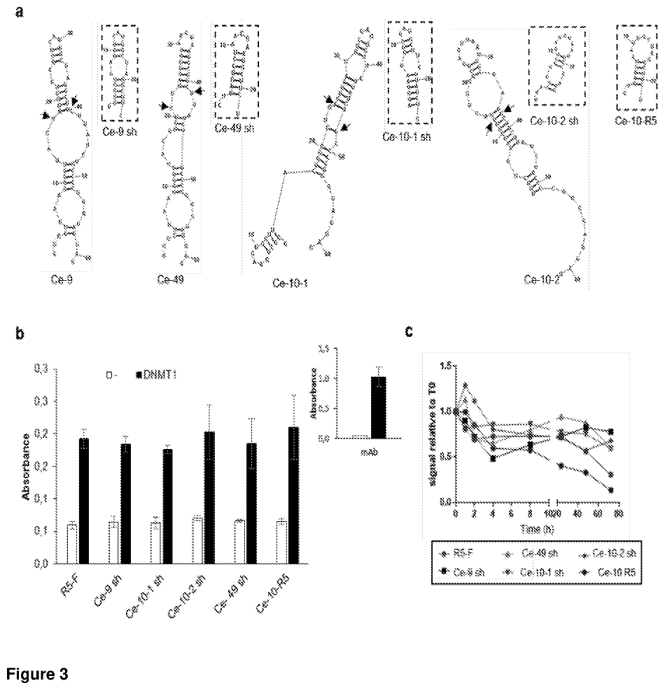

A key aspect of aptamer optimization is the reduction to a length compatible with an effective chemical synthesis. Thus, the possibility to truncate Ce-9, Ce-10 and Ce-49 aptamers were addressed to identify shortened versions, sufficient for full binding activity. Basing on the predicted secondary structures of the long sequences (61-mer), the present disclosure identified one truncated candidate for either Ce-9 or Ce-49 (indicated as Ce-9 sh and Ce-49 sh, respectively), and two alternative short sequences (Ce10-1 sh, Ce10-2 sh). These shortened aptamers cover the stem loop portions of the predicted structures without the 5′ and the 3′regions. Further, the present disclosure designed an additional short version consisting of the central linear region of Ce-10 (indicated as Ce-10-R5) also forming a stem and loop structure (FIG. 3a).

By analyzing the binding by ELONA assay at 200 nM concentration, the present inventors found that all the short aptamers are able to bind the purified DNMT1 at comparable levels (FIG. 3b), thus suggesting that the presence of the stem and loop is the key element required for DNMT1 binding.

The isolated sequences contains 2′F-Py in order to increase the resistance to enzymatic degradation providing a stable and easy to handle tool. Their serum stability was thus analyzed by incubating R5-F or the aptamers in 85% human serum for increasing times at 37° C. The present inventors found that all the sequences show a good serum stability remaining completely stable up to 48 hours. Ce-9 sh and Ce-10-2 sh revealed to be the most stable sequences getting only 20-30% degradation following 72 hours, whereas Ce-49 sh and Ce-10-1 sh reach about 40% and Ce-10 R5 and R5-F about 70-90%.

### DNMT1-Specific Aptamers: Binding Affinity and Specificity

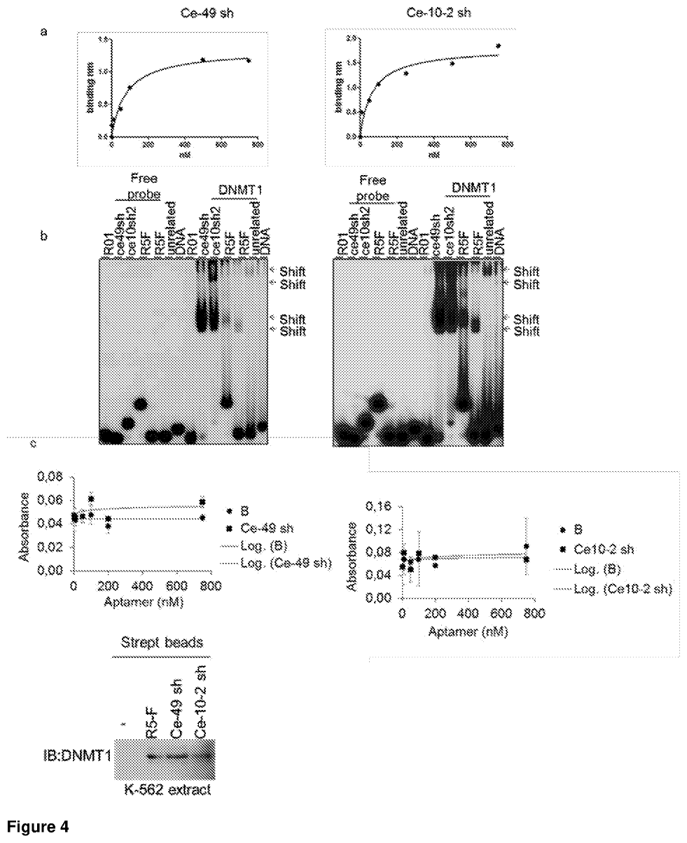

Next, aptamer affinity to DNMT1 was assessed by the Blitz system (ForteBIO). Among the tested sequences, the best affinity was detected for Ce-49 sh and Ce-10-2 sh showing a Kd of about 79 and 66 nM, respectively (FIG. 4a) as compared to an unstructured circular oligonucleotide (Mut-R5) used as a control. Kd was not measurable by this assay for R5-F, suggesting that the chosen sequences (Ce-49 sh and Ce-10-2 sh) greatly improved R5 binding to DNMT1. This result was confirmed by EMSA analyses (FIG. 4b).

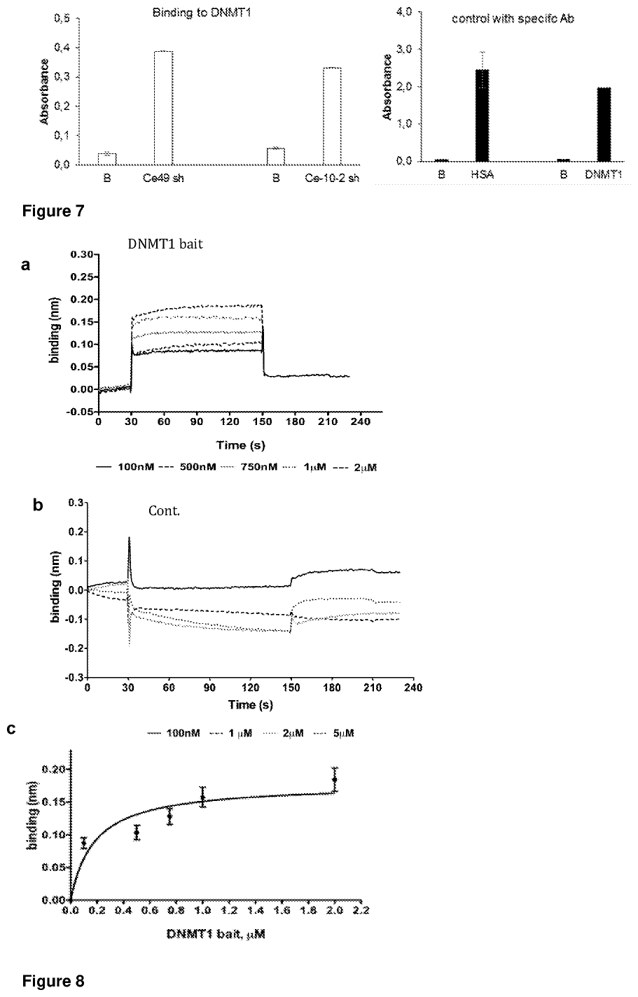

To check the aptamer specificity, the present disclosure monitored the binding of Ce-49 sh and Ce-10-2 sh to the human serum albumin (HSA), the most enriched plasma protein that can bind, in a nonspecific manner, nucleic acids through its positive charge, thus limiting their use. To this purpose, the present disclosure performed ELONA assay by incubation increasing concentration of Ce-49 sh and Ce-10-2 sh on plates previously coated or not-coated with HSA (FIG. 4c). As shown, no significant aptamer binding was measured up to 750 nM concentration. Binding was instead detected when aptamers were incubated with DNMT1 protein (FIG. 7).

Further, in order to demonstrate aptamer ability to bind not only the purified protein, but also the endogenous DNMT1, the present inventors performed an aptamer-mediated pull-down assay. To this end, extract from chronic myelogenous leukemia (CML) K562 cells showing high levels of DNMT1 were incubated with biotin-tagged R5-F, Ce-49 sh and Ce-10-2 sh, purified on streptavidin-coated beads, followed by immunoblotting with anti-DNMT1 antibody. As shown in FIG. 4d, aptamers interact with DNMT1.

Taken together, these data demonstrate that the generated short RNA aptamers: Ce-49 sh and Ce-10-2 sh showed an improved binding to DNMT1.

### Aptamer Functional Characterization

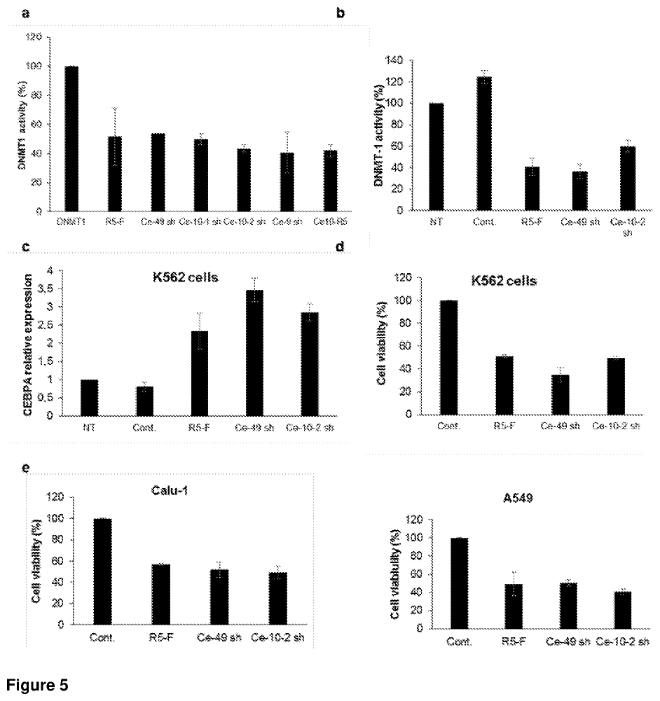

As next step, the present disclosure tested aptamer ability to inhibit DNMT1-mediated methylation. As first attempt, the present disclosure performed an in vitro DNMT1 inhibitor screening assay. The activity of DNMT1 purified protein was measured in the absence or in the presence of R5-F or the selected short sequences. As shown in FIG. 5a, all the sequence reduced DNMT1 function of about 40%. The functional effects of the best binders (Ce-49 sh and Ce-10-2 sh) was further characterized monitoring the in vivo DNMT1 activity by using cell nuclear extracts from chronic K562 CML cells transfected with the aptamers (FIG. 5b). Notably, a reduction of about 40-60% was detected upon aptamer transfection as compared to untreated cells and cells transfected with Mut-R5 oligo (indicated as Cont.).

Since ecCEBPA DiR induction leads to demethylation and increased in CEBPA mRNA expression, the present disclosure measured the effect of DNMT1-specific aptamers on CEBPA levels. Aptamers transfection resulted in an effective increase of CEBPA levels (FIG. 5c) in K562 cell line that express almost undetectable levels of ecCEBPA and CEBPA mRNA (Di Ruscio et al. Nature 2013). No effect was detected transfecting the cells with the control oligonucleotide.

Further, as compared with the control, K562 cell viability was reduced of about 60-50% upon aptamer transfection (FIG. 5d). The effect on cell viability was confirmed on non-small cell lung cancer (NSCLC) Calu-1 cell lines, confirming the potential therapeutic applicability of the selected sequences (FIG. 5e).

In conclusion, the present disclosure isolated a panel of DNMT1-specific RNA aptamers—with high stability, able to inhibit DNMT1 activity.

### Additional Experimental Data

Dose-response binding analyses were also performed with the 2′F-pyrimidine modified R5 (indicated as DNMT1-bait) at concentrations ranging between 100 nM and 2 μM, since no binding was detected at lower concentrations (FIG. 8a). In accordance with previous findings, no binding was detected for the mutR5 sequence which is unable to fold in stem-loop-like structures and used as a negative control (FIG. 8b). Data fitting (FIG. 8c) provided a KD value of 0.6±0.1*10⁻⁶ M (R2=0.9017), about 10 times higher as compared to that of the new molecules, a difference that further affirms the increased affinity of the two newly generated aptamers.

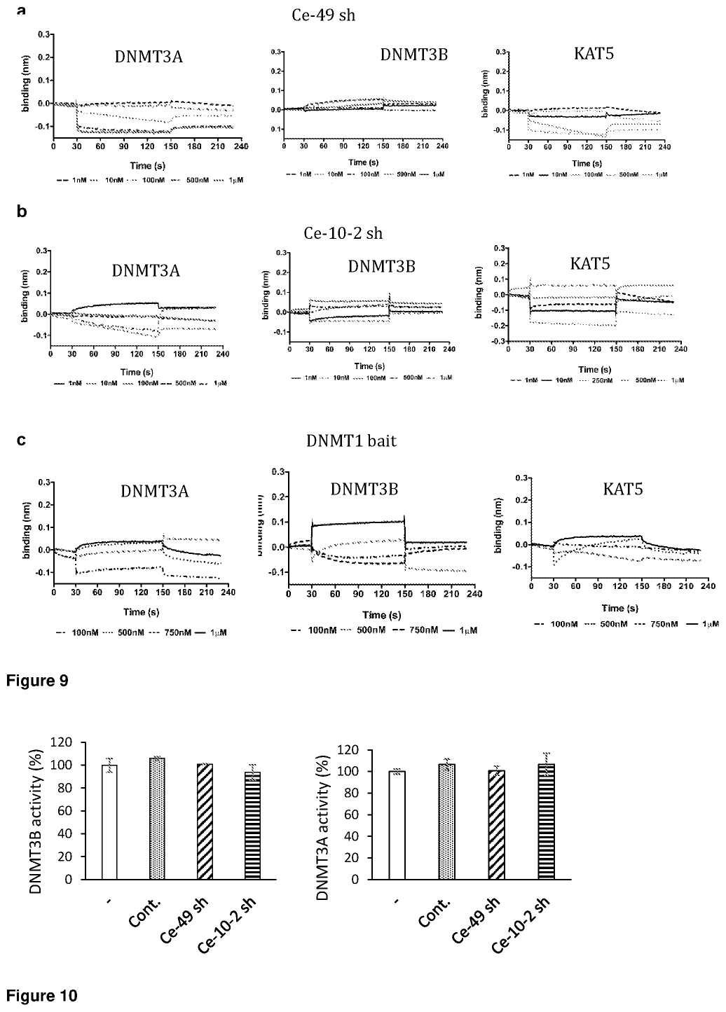

In order to evaluate the DNMT1 selectivity and specificity of the selected aptamers Ce-49 sh and Ce-10-2 sh, comparative binding experiments were performed with the other main members of the DNMT family: DNMT3A and DNMT3B and with the unrelated chromatin modifier lysine acetyltransferase 5 (KAT5). No significant interaction was recorded between the tested aptamers (FIG. 9a, b), that show the same specificity as DNMT1 bait, and the three control proteins (FIG. 9c).

The specificity was confirmed also by functional assay. The in vitro enzymatic activity of DNMT3A or DNMT3B purified proteins were measured in the absence or presence of 2′-F-Py R5 (DNMT1-bait) or the selected aptamers. The inventors found that the DNMT1-bait and Ce-49 sh and Ce-10-2 sh were unable to interfere in vitro with the enzymatic activity of DNMT3A/B (FIG. 10), thus confirming the high affinity and selectivity of the aptamers for DNMT1.

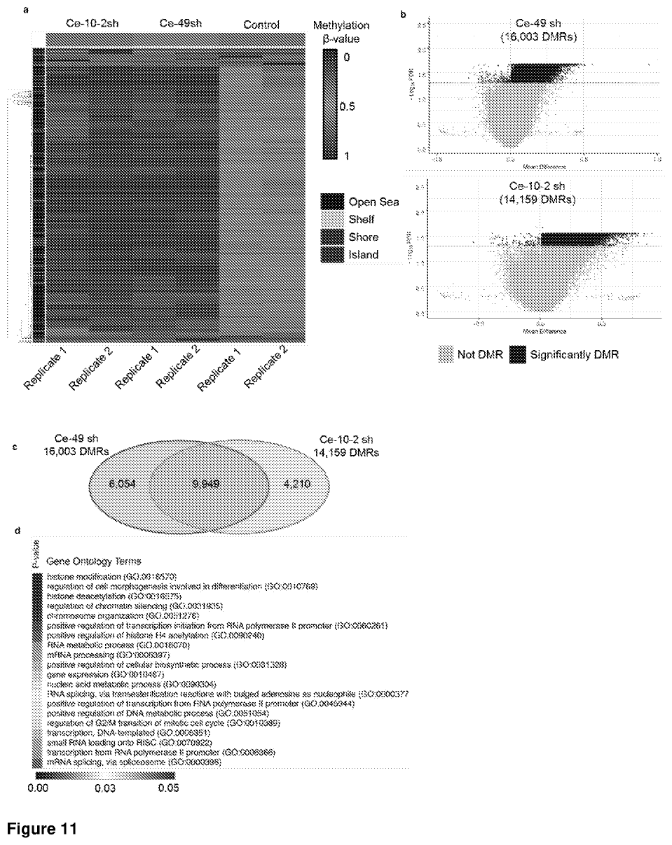

To estimate the extent of the effect on DNA methylation resulting from the DNMT1-specific aptamers, the genome-scale methylome profile was assessed by the EPIC array platform on K562 cells transfected with Ce-49 sh and Ce-10-2 sh. The differential methylation analyses revealed significant reduction of DNA methylation across thousands of CpG covered by the array in the aptamer-treated cells as compared to the control (FIG. 11a). Nearly 16,000 and 14,000 differentially methylated regions (DMRs) were detected for the Ce-49 sh and Ce-10-2 sh samples, respectively (FIGS. 11b and c) with more than 60% overlap between the two (FIG. 11c). Gene ontology (GO) analyses for “biological process” of genes corresponding to the overlapping hypo-methylated CpGs included among the top ranked, GO terms belonging to epigenetic modification, regulation of transcription and gene expression, consistently with the aptamer function (FIG. 11d).

The present disclosure describes an innovative protocol to isolate modified aptamers-based RNAs targeting DNMT1. By such an approach, different molecules with improved affinity for DNMT1 were provided. To enable a more efficient and cost-effective chemical synthesis, these sequences were optimized by designing shorter sequences that preserve the binding ability and display a very high serum stability. Further, we found that the selected aptamers are able to bind at high affinity to and specifically inhibit DNMT1 activity both in vitro and in cell cultures and impair cell viability in various cancer cell lines. In conclusion DNMT1-specific aptamers represent a promising tool to achieve specific inhibition of DNMT1 and hold promise for a genuine and targeted therapy with broad clinical applicability.

1. An aptamer capable of inhibiting DNA methyltransferase 1 (DNMT1), wherein the aptamer comprises a stem loop structure deriving from DiR:ecCEBPA that is capable of interacting with DNMT1 to thereby inhibit DNMT1.

2. The aptamer of claim 1, wherein
the aptamer is capable of reducing DNMT1 function by at least about 30%, and/or
the aptamer is capable of increasing CEBPA (CCAAT/enhancer-binding protein alpha) levels, and/or the aptamer is capable of reducing the viability of a cancerous cell.

3. The aptamer of claim 1, wherein
the aptamer comprises a stem structure comprising two or more pairs of nucleotides, and/or
the aptamer comprises a loop structure formed by four or more nucleotides.

4. The aptamer of any claim 1, wherein the aptamer is an RNA-aptamer.

5. The aptamer of claim 1, wherein the aptamer comprises between 10 to 61 nucleotides, and/or the aptamer further comprises a modification capable of enhancing nuclease resistance, optionally the modification is a 2′-fluoro-, 2′-methoxy-, 2′-methoxyethyl-, and/or 2,-amino-modified nucleotides, optionally the aptamer comprises 2′-Fluoro-Pyrimidines (2′F-Py) modification.

6. The aptamer of claim 1, wherein
the aptamer comprises a nucleotide sequence that is at least 70% identical to any one of sequences shown in FIG. 6, optionally
the aptamer comprises a nucleotide sequence having at least 70% sequence identity to sequences selected from the group consisting of 5′ CUGAGCUCAUGGCGAGGCUUCU 3′ (SEQ ID NO: 9), 5′ UGGGCUGAGCUCAUGGCGAGGCUUC 3′ (SEQ ID NO: 67), 5′ CUGAGGCCUAACGAAGGCUUCU 3′ (SEQ ID NO: 68), 5′ CUGAGGCCUAACGAAGGCUUCU 3′ (SEQ ID NO: 68), 5′ CUGAGGUAAUGGCGAGGCUUCU 3′ (SEQ ID NO: 69), 5′ AGGUAAUGGCGAGGCUUCUUAUCUG 3′ (SEQ ID NO: 70), 5′ UUACUGGGCUGAGGUAAUGGCGAGG 3′ (SEQ ID NO: 71), and 5′ CTGAGGTAATGGCGAGGCTTCT 3′ (SEQ ID NO: 72).

7. (canceled)

8. The aptamer of claim 1 wherein the aptamer is comprised in a pharmaceutical composition.

9. A method of treating or preventing a disease characterized by aberrant DNA methylation in a subject in need thereof, the method comprising:
administering an effective amount of the aptamer of claim 1 to modulate DNA methylation in the subject in need thereof.

10. (canceled)

11. The method of claim 9, wherein the method comprises:
administering the aptamer of claim 1 to thereby inhibit DNMT1 activity in the subject in need thereof, optionally
wherein the subject has a condition/disease characterized by changes in DNA methylation or aberrant DNA methylation that may include, but is not limited to, cancer, autoimmune diseases, genetic disorders, metabolic disorders, psychological disorders, and aging, optionally
the disease characterized by aberrant DNA methylation is chronic myelogenous leukemia (CML), and/or non-small cell lung cancer (NSCLC), including adenocarcinoma (such as human alveolar basal epithelial adenocarcinoma) and squamous cell carcinoma.

12. A method of producing and/or selecting inhibitor(s) of a DNA methyltransferase, the method comprising:
preparing one or more libraries of variants by introducing one or more alterations in an aptamer sequence capable of interacting with the DNA methyltransferase, wherein the one or more alterations is in a central region of the aptamer sequence and/or introduces a 2′-fluoro-pyrimidines modification;
contacting/incubating the variants with a target DNA methyltransferase to allow the variants to bind to the target DNA methyltransferase;
separating the variant(s) bound to the target DNA methyltransferase from the unbound variant(s); and
recovering the variant(s) bound to the target DNA methyltransferase to obtain the inhibitor(s) of the DNA methyltransferase.

13. The method of claim 12, wherein
the preparing one or more libraries of variants further comprises adding a primer sequence to the variant, optionally
the preparing one or more libraries of variants further comprises adding a promoter to the variant, optionally the variant comprises a stem and loop structure.

14. The method of claim 13,
wherein preparing the one or more libraries of variants comprises preparing sub-libraries of variants by introducing the one or more alterations in different pre-determined regions within the central region to form different sub-libraries of variants having alterations in different pre-determined regions.

15. The method of claim 14,
wherein the variants comprise one or more flanking regions that are free of alteration.

16. The method of claim 12, wherein the method comprises mixing variants from each sub-library to form a diverse pool of variants for the contacting/incubating step.

17. The method of claim 12, wherein the method further comprises truncating the variants to obtain shortened variants retaining a stem and loop structure.

18. The method of claim 12, wherein the DNA methyltransferase comprises DNMT1.

19. The method of claim 12, wherein the one or more alterations is a randomization of the sequence and/or a 2′-fluoro-pyrimidines modification.


SEQUENCE LISTING (74 sequences)

SEQ ID NO: 1 - DNA, 22 bp, Artificial sequence
  Synthetic sequence
cugagcgacuggcgaggcuucu

SEQ ID NO: 2 - DNA, 22 bp, Artificial sequence
  Synthetic sequence
cugaguugcuggcgaggcuucu

SEQ ID NO: 3 - DNA, 22 bp, Artificial sequence
  Synthetic sequence
cugaguauauggcgaggcuucu

SEQ ID NO: 4 - DNA, 22 bp, Artificial sequence
  Synthetic sequence
cugaggccuacacuaggcuucu

SEQ ID NO: 5 - DNA, 22 bp, Artificial sequence
  Synthetic sequence
cugaggccuuggcgaggcuucg

SEQ ID NO: 6 - DNA, 22 bp, Artificial sequence
  Synthetic sequence
cugagcccuuggcgaggcuucc

SEQ ID NO: 7 - DNA, 22 bp, Artificial sequence
  Synthetic sequence
cugaggccugcaguaggcuucu

SEQ ID NO: 8 - DNA, 22 bp, Artificial sequence
  Synthetic sequence
cugaguaacuggcgaggcuucu

SEQ ID NO: 9 - DNA, 22 bp, Artificial sequence
  Synthetic sequence
cugagcucauggcgaggcuucu

SEQ ID NO: 10 - DNA, 22 bp, Artificial sequence
  Synthetic sequence
cugagguaauggcgaggcuucu

SEQ ID NO: 11 - DNA, 22 bp, Artificial sequence
  Synthetic sequence
cugaggccuuggcgaggcgacc

SEQ ID NO: 12 - DNA, 22 bp, Artificial sequence
  Synthetic sequence
cugaggccuuggcgaggcggac

SEQ ID NO: 13 - DNA, 22 bp, Artificial sequence
  Synthetic sequence
cugaggccuuggcgaggcugac

SEQ ID NO: 14 - DNA, 22 bp, Artificial sequence
  Synthetic sequence
cugagaacuuggcgaggcuucu

SEQ ID NO: 15 - DNA, 22 bp, Artificial sequence
  Synthetic sequence
cugaggccuuggcgaggccguc

SEQ ID NO: 16 - DNA, 22 bp, Artificial sequence
  Synthetic sequence
cugaggccuuggcgaggcucgu

SEQ ID NO: 17 - DNA, 22 bp, Artificial sequence
  Synthetic sequence
cugaggccugguggaggcuucu

SEQ ID NO: 18 - DNA, 22 bp, Artificial sequence
  Synthetic sequence
cugaguuauuggcgaggcuucu

SEQ ID NO: 19 - DNA, 22 bp, Artificial sequence
  Synthetic sequence
cugaggccuuggcgaggccacg

SEQ ID NO: 20 - DNA, 22 bp, Artificial sequence
  Synthetic sequence
cugagcucauggcgaggcuucu

SEQ ID NO: 21 - DNA, 22 bp, Artificial sequence
  Synthetic sequence
cugaggccuacccaaggcuucu

SEQ ID NO: 22 - DNA, 22 bp, Artificial sequence
  Synthetic sequence
cugaggccuauguaaggcuucu

SEQ ID NO: 23 - DNA, 22 bp, Artificial sequence
  Synthetic sequence
cugaggccuaugugaggcuucu

SEQ ID NO: 24 - DNA, 22 bp, Artificial sequence
  Synthetic sequence
cugaggccucagcuaggcuucu

SEQ ID NO: 25 - DNA, 22 bp, Artificial sequence
  Synthetic sequence
cugagcaacuggcgaggcuucu

SEQ ID NO: 26 - DNA, 22 bp, Artificial sequence
  Synthetic sequence
cugagugccuggcgaggcuucu

SEQ ID NO: 27 - DNA, 22 bp, Artificial sequence
  Synthetic sequence
cugaggcauuggcgaggcuucu

SEQ ID NO: 28 - DNA, 22 bp, Artificial sequence
  Synthetic sequence
cugaggccugcccuaggcuucu

SEQ ID NO: 29 - DNA, 22 bp, Artificial sequence
  Synthetic sequence
cugaggccuuggcgaggcaccc

SEQ ID NO: 30 - DNA, 22 bp, Artificial sequence
  Synthetic sequence
cugagaaccuggcgaggcuucc

SEQ ID NO: 31 - DNA, 22 bp, Artificial sequence
  Synthetic sequence
cugaggccuaugguaggcuucu

SEQ ID NO: 32 - DNA, 22 bp, Artificial sequence
  Synthetic sequence
cugagugcuuggcgagacuucu

SEQ ID NO: 33 - DNA, 22 bp, Artificial sequence
  Synthetic sequence
cugaggccuugucuaggcuucu

SEQ ID NO: 34 - DNA, 22 bp, Artificial sequence
  Synthetic sequence
cugaggccuagccaaggcuucu

SEQ ID NO: 35 - DNA, 22 bp, Artificial sequence
  Synthetic sequence
cugaggccuuggcgaggcuccc

SEQ ID NO: 36 - DNA, 22 bp, Artificial sequence
  Synthetic sequence
cugaggccugaacaaggcuucu

SEQ ID NO: 37 - DNA, 22 bp, Artificial sequence
  Synthetic sequence
cugaggucuugucgaggcaucc

SEQ ID NO: 38 - DNA, 22 bp, Artificial sequence
  Synthetic sequence
cugagaccguggcgaggcuucu

SEQ ID NO: 39 - DNA, 22 bp, Artificial sequence
  Synthetic sequence
cugaggccuuggcgaggcaacg

SEQ ID NO: 40 - DNA, 22 bp, Artificial sequence
  Synthetic sequence
cugaggccuuggcgaggcaucc

SEQ ID NO: 41 - DNA, 22 bp, Artificial sequence
  Synthetic sequence
cugaggccuccauaaggcuucu

SEQ ID NO: 42 - DNA, 22 bp, Artificial sequence
  Synthetic sequence
cugaggccuagcacaggcuucu

SEQ ID NO: 43 - DNA, 22 bp, Artificial sequence
  Synthetic sequence
cugaggccugccguaggcuucu

SEQ ID NO: 44 - DNA, 22 bp, Artificial sequence
  Synthetic sequence
cugaggccugggucaggcuucu

SEQ ID NO: 45 - DNA, 22 bp, Artificial sequence
  Synthetic sequence
cugaggccuaacgaaggcuucu

SEQ ID NO: 46 - DNA, 22 bp, Artificial sequence
  Synthetic sequence
cugagaccuuggcgaggcuucc

SEQ ID NO: 47 - DNA, 22 bp, Artificial sequence
  Synthetic sequence
cugagguaauggcgaggcuucu

SEQ ID NO: 48 - DNA, 22 bp, Artificial sequence
  Synthetic sequence
cugagcagguggcgaggcuucu

SEQ ID NO: 49 - DNA, 22 bp, Artificial sequence
  Synthetic sequence
cugaggccugagucaggcuucu

SEQ ID NO: 50 - DNA, 22 bp, Artificial sequence
  Synthetic sequence
cugaggccuuggcgaggcgccc

SEQ ID NO: 51 - DNA, 22 bp, Artificial sequence
  Synthetic sequence
cugaggacauggcgaggcuucu

SEQ ID NO: 52 - DNA, 22 bp, Artificial sequence
  Synthetic sequence
cugaggccuuggcgaggcacgu

SEQ ID NO: 53 - DNA, 22 bp, Artificial sequence
  Synthetic sequence
cugaggccugauguaggcuucu

SEQ ID NO: 54 - DNA, 22 bp, Artificial sequence
  Synthetic sequence
cugagcccguggcgaggcuucu

SEQ ID NO: 55 - DNA, 22 bp, Artificial sequence
  Synthetic sequence
cugaggccuuggcgaggcucug

SEQ ID NO: 56 - DNA, 22 bp, Artificial sequence
  Synthetic sequence
cugaggccuuggcgaggcgcug

SEQ ID NO: 57 - DNA, 22 bp, Artificial sequence
  Synthetic sequence
cugaggccuuggcgaggccugg

SEQ ID NO: 58 - DNA, 22 bp, Artificial sequence
  Synthetic sequence
cugaggccuuggcgaggcccgc

SEQ ID NO: 59 - DNA, 22 bp, Artificial sequence
  Synthetic sequence
cugaggccuuggcgaggcuugu

SEQ ID NO: 60 - DNA, 22 bp, Artificial sequence
  Synthetic sequence
cugaguaccuggcgaggcuucu

SEQ ID NO: 61 - DNA, 22 bp, Artificial sequence
  Synthetic sequence
cugaggccuuggcgaggccaaa

SEQ ID NO: 62 - DNA, 22 bp, Artificial sequence
  Synthetic sequence
cugagaagauggcgaggcuucu

SEQ ID NO: 63 - DNA, 22 bp, Artificial sequence
  Synthetic sequence
cugaggccuuggcgaggcaguu

SEQ ID NO: 64 - DNA, 22 bp, Artificial sequence
  Synthetic sequence
cugaggccuacuggaggcuucu

SEQ ID NO: 65 - DNA, 22 bp, Artificial sequence
  Synthetic sequence
cugaggccuagcaaaggcuucu

SEQ ID NO: 66 - DNA, 22 bp, Artificial sequence
  Synthetic sequence
cugaggccuuggcgaggcuucu

SEQ ID NO: 67 - DNA, 25 bp, Artificial Sequence
  Synthetic sequence
ugggcugagcucauggcgaggcuuc

SEQ ID NO: 68 - DNA, 22 bp, Artificial Sequence
  Synthetic sequence
cugaggccuaacgaaggcuucu

SEQ ID NO: 69 - DNA, 22 bp, Artificial sequence
  Synthetic sequence
cugagguaauggcgaggcuucu

SEQ ID NO: 70 - DNA, 25 bp, Artificial sequence
  Synthetic sequence
agguaauggcgaggcuucuuaucug

SEQ ID NO: 71 - DNA, 25 bp, Artificial sequence
  Synthetic sequence
uuacugggcugagguaauggcgagg

SEQ ID NO: 72 - DNA, 22 bp, Artificial sequence
  Synthetic sequence
ctgaggtaatggcgaggcttct

SEQ ID NO: 73 - DNA, 37 bp, Artificial sequence
  Synthetic sequence
taatacgactcactatagggctgaaggggttactggg

SEQ ID NO: 74 - DNA, 19 bp, Artificial sequence
  Synthetic sequence
ctcctccccggggcagata